(19) United States
(12) Patent Application Publication
(10) Pub. No.: US 2009/0123924 A1
(43) Pub. Date: May 14, 2009

(54) Method for Breast Cancer Diagnosis

(75) Inventors: Alexander Krause, Lyon (FR); Philippe Leissner, Lyon (FR); Bruno Mougin, Lyon (FR); Malick Paye, Grenoble (FR)

Correspondence Address: OLIFF & BERRIDGE, PLC, P.O. BOX 320850, ALEXANDRIA, VA, 22320-4850, US

(73) Assignee: Biomerieux, Marcy L'Etoile (FR)

(21) Appl. No.: 11/988,364

(22) PCT Filed: Jul. 5, 2006
(86) PCT No.: PCT/FR2006/001593

§ 371 Date: Jan. 7, 2008

(30) Foreign Application Priority Data
Jul. 7, 2005 (FR) 0552087

(51) Int. Cl. C12Q 1/68; C07H 21/04

(52) U.S. Cl. 435/6; 536/24.31

(57) ABSTRACT

The invention relates to a method for the in vitro diagnosis of breast cancer in a patient who may be suffering from a breast cancer, characterized in that it comprises the following steps:
a) biological material is extracted from a biological sample taken from the patient,
b) the biological material is brought into contact with at least 8 specific reagents chosen from the specific reagents for the target genes with a nucleic sequence having any one of SEQ ID Nos. 1 to 8,
c) the expression of said target genes is determined.

The present invention relates to the cancerology field. More particularly, the invention relates to a method for breast cancer diagnosis.

In women, breast cancer is the leading cause of mortality due to cancer in industrialized countries. Age is the most important risk factor. Thus, the risk increases by 0.5% per year of age in countries in the West. Other risk factors are known, such as the number of pregnancies and the age at the time of the first pregnancy, breast-feeding, the age at puberty and at the menopause, estrogenic treatments after the menopause has occurred, stress and nutrition.

Breast cancer diagnosis is generally carried out by mammography. However, it is estimated that the minimum size of a tumor that can be detected by mammography is 1 cm, which means that the tumor has an evolutive past of 8 years, on average, at the time of diagnosis. In addition, the early detection of a tumor is all the more important because small tumors are much less malignant than what can be extrapolated from their sizes: the aggressiveness of large tumors does not come only from their size, but also from their “inherent aggressiveness”, which increases with the age of a tumor (Bucchi et al., Br J Cancer 2005, p. 156-161; Norden T, Eur J Cancer 1997, p. 624-628). The analysis of the expression of a panel of target genes is also relevant in combating breast cancer, and mention may in particular be made of the analysis of a panel of 176 genes, which is expressed differentially between patients expressing the ER receptor and patients not expressing the ER receptor (Bertucci et al., Human Molecular Genetics, 2000; 9: 2981-2991). Mention may also be made of the analysis of a panel of 37 genes which makes it possible to make an early diagnosis of the breast cancer (Sharma et al., Breast cancer Research 2005, 7:R634—R644). However, all the patients had a suspect initial mammogram. This gene panel could be unsuitable for a routine diagnostic test for breast cancer prior to any mammography.

It is therefore very useful to have a tool for making a very early breast cancer diagnosis.

The present invention proposes a novel method for breast cancer diagnosis, which makes it possible to distinguish patients suffering from a breast cancer very early, but also at an advanced stage. The analysis of the expression of these target genes can be carried out directly using a blood sample, which makes it possible to avoid serious or detrimental procedures, and the uncertainties associated with the taking of a sample during these procedures. Since peripheral blood is the most clinically accessible interior compartment, the use of this source in particular allows a routine diagnosis, which is less restricting than mammography, to be carried out.

Before proceeding with the disclosure of the invention, the following definitions, which apply for all the variants of the invention, should be given.

For the purpose of the present invention, the term “biological sample” is intended to mean any sample taken from a patient and liable to contain a biological material as defined hereinafter. This biological sample may in particular be a blood, serum, saliva, tissue, tumor or bone marrow sample or a sample of circulating cells from the patient. This biological sample is obtained by any means for taking a sample known to those skilled in theart.

For the purpose of the present invention, the term “biological material” is intended to mean any material which makes it possible to detect the expression of a target gene. The biological material may in particular comprise proteins, or nucleic acids such as, in particular, deoxyribonucleic acids (DNA) or ribonucleic acids (RNA). The nucleic acid may in particular be an RNA (ribonucleic acid). According to a preferred embodiment of the invention, the biological material comprises nucleic acids, preferably RNA, and even more preferably total RNA. Total RNA comprises transfer RNA, messenger RNA (mRNA), such as the mRNA transcribed from the target gene, but also transcribed from any other gene, and ribosomal RNA. This biological material comprises material specific for a target gene, such as in particular the mRNA transcribed from the target gene or the proteins derived from this mRNA, but may also comprise material not specific for a target gene, such as in particular the mRNA transcribed from a gene other than the target gene, the tRNA, or the rRNA derived from genes other than the target gene.

During step a) of the method according to the invention, the biological material is extracted from the biological sample by any of the protocols for extracting and purifying nucleic acids well known to those skilled in the art. By way of indication, the nucleic acid extraction can be carried out by means of:
a step for lysis of the cells present in the biological sample, in order to release the nucleic acids contained in the cells from the patient. By way of example, the lysis methods as described in patent applications WO 00/05338 (mixed magnetic and mechanical lysis), WO 99/53304 (electrical lysis) and WO 99/15321 (mechanical lysis) can be used. Those skilled in the art may use other well known methods of lysis, such as thermal or osmotic shocks or chemical lysis with chaotropic agents such as guanidinium salts (U.S. Pat. No. 5,234,809).
a purification step which makes it possible to separate the nucleic acids from the other cellular constituents released during the lysis step. This step generally makes it possible to concentrate the nucleic acids and can be adapted to the purification of DNA or RNA. By way of example, use may be made of magnetic particles optionally coated with oligonucleotides, by adsorption or covalence (see, in this respect, U.S. Pat. No. 4,672,040 and U.S. Pat. No. 5,750,338), and the nucleic acids which are bound to these magnetic particles can thus be purified by means of a washing step. This nucleic acid purification step is particularly advantageous if it is desired to subsequently amplify said nucleic acids. A particularly advantageous embodiment of these magnetic particles is described in patent applications: WO-A-97/45202 and WO-A-99/35500. Another advantageous example of a nucleic acid purification method is the use of silica, either in the form of a column, or in the form of inert particles (Boom R. et al., J. Clin. Microbiol., 1990, n^{o} 28(3), p. 495-503) or magnetic particles (Merck: MagPrep® Silica, Promega: MagneSil™ Paramagnetic particles). Other very widely used methods are based on ion exchange resins in a column or in a paramagnetic particulate format (Whatman: DEAE-Magarose) (Levison P R et al., J. Chromatography, 1998, p. 337-344). Another method which is very relevant but not exclusive for the invention is that of adsorption onto a metal oxide support (from the company Xtrana: Xtra-Bind™ matrix). When it is desired to specifically extract the DNA from a biological sample, an extraction with phenol, chloroform and alcohol can in particular be carried out in order to remove the proteins, and the DNA can be precipitated with 100% ethanol. The DNA can then be pelleted by centrifugation, washed and redissolved. When it is desired to specifically extract the RNA from a biological sample, an extraction with phenol, chloroform and alcohol can in particular be carried out in order to remove the proteins and the RNA can be precipitated with 100% ethanol.

The RNA can then be pelleted by centrifugation, washed and redissolved. During step b, and for the purposes of the present invention, the term “specific reagent” is intended to mean a reagent which, when it is brought into contact with biological material as defined above, binds with the material specific for said target gene. By way of indication, when the specific reagent and the biological material are of nucleic origin, bringing the specific reagent into contact with the biological material allows the specific reagent to hybridize with the material specific for the target gene. The term “hybridization” is intended to mean the process during which, under suitable conditions, two nucleotide fragments bind with stable and specific hydrogen bonds so as to form a double-stranded complex. These hydrogen bonds form between the complementary adenine (A) and thymine (T) (or uracil (U)) bases (this is then referred to as an A-T bond) or between the complementary guanine (G) and cytosine (C) bases (this is then referred to as a G-C bond). The hybridization of two nucleotide fragments may be total (reference is then made to complementary nucleotide fragments or sequences), i.e. the double-stranded complex obtained during this hybridization comprises only A-T bonds and C-G bonds. This hybridization may be partial (reference is then made to sufficiently complementary nucleotide fragments or sequences), i.e. the double-stranded complex obtained comprises A-T bonds and C-G bonds allowing the double-stranded complex to form, but also bases not bonded to a complementary base. The hybridization between two nucleotide fragments depends on the working conditions which are used, and in particular on the stringency. The stringency is defined in particular according to the base composition of the two nucleotide fragments, and also by the degree of mismatching between two nucleotide fragments. The stringency may also depend on the reaction parameters, such as the concentration and the type of ionic species present in the hybridization solution, the nature and the concentration of denaturing agents and/or the hybridization temperature. All these data are well known and the appropriate conditions can be determined by those skilled in the art. In general, according to the length of the nucleotide fragments that it is desired to hybridize, the hybridization temperature is between approximately 20 and 70° C., in particular between 35 and 65° C., in a saline solution at a concentration of approximately 0.5 to 1 M. A sequence, or nucleotide fragment, or oligonucleotide, or polynucleotide is a series of nucleotide motifs assembled together by phosphoric ester bonds, characterized by the informational sequence of the natural nucleic acids, capable of hybridizing to a nucleotide fragment, it being possible for the series to contain monomers of different structures and to be obtained from a natural nucleic acid molecule and/or by genetic recombination and/or by chemical synthesis. A motif is derived from a monomer which may be a natural nucleotide of a nucleic acid, the constitutive elements of which are a sugar, a phosphate group and a nitrogenous base; in DNA, the sugar is deoxy-2-ribose, in RNA, the sugar is ribose; depending on whether it is a question of DNA or RNA, the nitrogenous base is chosen from adenine, guanine, uracil, cytosine and thymine; or else the monomer is a nucleotide modified in at least one of the three constitutive elements: by way of example, the modification may occur either at the level of the bases, with modified bases such as inosine, methyl-5-deoxycytidine, deoxyuridine, dimethylamino-5-deoxyuridine, diamino-2,6-purine, bromo-5-deoxyuridine or any other modified base capable of hybridization, or at the level of the sugar, for example the replacement of at least one deoxyribose with a polyamide (P. E. Nielsen et al., Science, 254, 1497-1500 (1991)), or else at the level of the phosphate group, for example replacement thereof with esters chosen in particular from diphosphates, alkyl and aryl phosphonates and phosphorothioates.

For the purpose of the present invention, the specific reagent is an amplification primer. For the purpose of the present invention, the term “amplification primer” is intended to mean a nucleotide fragment comprising from 5 to 100 nucleic motifs, preferably from 15 to 30 nucleic motifs, allowing the initiation of an enzymatic polymerization, such as, in particular, an enzymatic amplification reaction. The term “enzymatic amplification reaction” is intended to mean a process which generates multiple copies of a nucleotide fragment through the action of at least one enzyme. Such amplification reactions are well known to those skilled in the art and mention may in particular be made of the following techniques:
PCR (polymerase chain reaction), as described in U.S. Pat. No. 4,683,195, U.S. Pat. No. 4,683,202 and U.S. Pat. No. 4,800,159,
LCR (ligase chain reaction), disclosed, for example, in patent application EP 0 201 184,
RCR (repair chain reaction), described in patent application WO 90/01069,
3SR (self sustained sequence replication) with patent application WO 90/06995,
NASBA (nucleic acid sequence-based amplification) with patent application WO 91/02818, and
TMA (transcription-mediated amplification) with U.S. Pat. No. 5,399,491.

When the enzymatic amplification is a PCR, the specific reagent comprises at least two amplification primers, specific for a target gene, which allow the amplification of the target gene-specific material. The target gene-specific material then preferably comprises a complementary DNA obtained by reverse transcription of messenger RNA derived from the target gene (reference is then made to target gene-specific cDNA) or a complementary RNA obtained by transcription of the cDNAs specific for a target gene (reference is then made to target gene-specific cRNA). When the enzymatic amplification is a PCR carried out after a reverse transcription reaction, this is referred to as RT-PCR.

The specific reagent may also be a hybridization probe. The term “hybridization probe” is intended to mean a nucleotide fragment comprising at least 5 nucleic motifs, preferably from 5 to 100 nucleic motifs, even more preferably from 10 to 35 nucleic motifs, having a hybridization specificity under given conditions so as to form a hybridization complex with the material specific for a target gene. In the present invention, the target gene-specific material may be a nucleotide sequence included in a messenger RNA derived from the target gene (reference is then made to target gene-specific mRNA), a nucleotide sequence included in a complementary DNA obtained by reverse transcription of said messenger RNA (reference is then made to target gene-specific cDNA), or else a nucleotide sequence included in a complementary RNA obtained by transcription of said cDNA as described above (reference will then be made to target gene-specific cRNA). The hybridization probe may comprise a label for its detection. The term “detection” is intended to mean either a direct detection by a physical method, or an indirect detection by a detection method using a label. Many detection methods exist for the detection of nucleic acids [see, for example, Kricka et al., Clinical Chemistry, 1999, n^{o} 45(4), p. 453-458 or Keller G. H. et al., DNA Probes, 2nd Ed., Stockton Press, 1993, sections 5 and 6, p. 173-249]. The term “label” is intended to mean a tracer capable of generating a signal that can be detected. A nonlimiting list of these tracers includes enzymes which produce a signal detectable, for example, by colorimetry, fluorescence or luminescence, such as horseradish peroxidase, alkaline phosphatase, beta-galactosidase or glucose-6-phosphate dehydrogenase; chromophores such as fluorescent, luminescent or dye compounds; electron dense groups detectable by electron microscopy or by their electrical properties such as conductivity, by amperometry or voltametry methods, or by impedance measurements; groups that can be detected by optical methods such as diffraction, surface plasmon resonance, contact angle variation or by physical methods such as atomic force spectroscopy, tunnel effect, etc.; radioactive molecules such as ³²P, ³⁵S or ¹²⁵I.

For the purpose of the present invention, the hybridization probe may be a “detection” probe. In this case, the “detection” probe is labeled with a label as defined above. The detection probe may in particular be a “molecular beacon” detection probe as described by Tyagi & Kramer (Nature Biotechnol., 1996, 14:303-308). These “molecular beacons” become fluorescent during hybridization. They have a stem-loop structure and contain a fluorophore and a quencher group. The binding of the specific loop sequence with its complementary target nucleic acid sequence causes the stem to uncoil and a fluorescent signal to be emitted during excitation at the appropriate wavelength.

For the detection of the hybridization reaction, use may be made of target sequences that have been labeled directly (in particular by the incorporation of a label within the target sequence) or indirectly (in particular using a detection probe as defined above) the target sequence. A step for labeling and/or cleaving the target sequence can in particular be carried out before the hybridization step, for example using a labeled deoxyribonucleotide triphosphate during the enzymatic amplification reaction. The cleavage can be carried out in particular through the action of imidazole and manganese chloride. The target sequence can also be labeled after the amplification step, for example by hybridizing a detection probe according to the sandwich hybridization technique described in document WO 91/19812. Another specific preferred method for labeling nucleic acids is described in application FR2780 059.

The hybridization probe may also be a “capture” probe. In this case, the “capture” probe is immobilized or immobilizable on a solid support by any appropriate means, i.e. directly or indirectly, for example by covalence or adsorption. As solid support, use may be made of synthetic materials or natural materials, optionally chemically modified, in particular polysaccharides such as cellulose-based materials, for example paper, cellulose derivatives such as cellulose acetate or nitrocellulose, or dextran, polymers, copolymers, in particular based on styrene-type monomers, natural fibers such as cotton, and synthetic fibers such as nylon; mineral materials such as silica, quartz, glasses, ceramics; latices; magnetic particles; metal derivatives, gels, etc. The solid support may be in the form of a microtitration plate, of a membrane as described in application WO-A-94/12670, or of a particle. Several different capture probes, each being specific for a target gene, can also be immobilized on the support. In particular, a biochip on which a large number of probes can be immobilized may be used as support. The term “biochip” is intended to mean a solid support which is small in size and to which are attached a multitude of capture probes at predetermined positions. The biochip, or DNA chip, concept dates from the beginning of the 1990s. It is based on a multidisciplinary technology which integrates microelectronics, nucleic acid chemistry, image analysis and information technology. The operating principle is based on a foundation of molecular biology: the hybridization phenomenon, i.e. the pairing, by complementarity, of the bases of two DNA and/or RNA sequences. The biochip method is based on the use of capture probes attached to a solid support, on which probes a sample of target nucleotide fragments directly or indirectly labeled with fluorochromes is made to act. The capture probes are positioned specifically on the substrate or chip and each hybridization gives a specific piece of information, in relation to the target nucleotide fragment. The pieces of information obtained are cumulative, and make it possible, for example, to quantify the level of expression of one or more target genes. In order to analyze the expression of a target gene, a biochip carrying a very large number of probes which correspond to all or part of the target gene, which is transcribed to mRNA, can then be prepared. The cDNAs or cRNAs specific for a target gene that it is desired to analyze are then hybridized, for example, on specific capture probes. After hybridization, the support or chip is washed and the labeled cDNA or cRNA/capture probe complexes are revealed by means of a high-affinity ligand bound, for example, to a fluorochrome-type label. The fluorescence is read, for example, with a scanner and the analysis of the fluorescence is processed by information technology. By way of indication, mention may be made of the DNA chips developed by the company Affymetrix (“Accessing Genetic Information with High-Density DNA arrays”, M. Chee et al., Science, 1996, 274, 610-614. “Light-generated oligonucleotide arrays for rapid DNA sequence analysis”, A. Caviani Pease et al., Proc. Natl. Acad. Sci. USA, 1994, 91, 5022-5026), for molecular diagnoses. In this technology, the capture probes are generally small in size, around 25 nucleotides. Other examples of biochips are given in the publications by G. Ramsay, Nature Biotechnology, 1998, n^{o} 16, p. 40-44; F. Ginot, Human Mutation, 1997, n^{o} 10, p. 1-10; J. Cheng et al., Molecular diagnosis, 1996, n^{o} 1(3), p. 183-200; T. Livache et al., Nucleic Acids Research, 1994, n^{o} 22(15), p. 2915-2921; J. Cheng et al., Nature Biotechnology, 1998, n^{o} 16, p. 541-546 or in U.S. Pat. No. 4,981,783, U.S. Pat. No. 5,700,637, U.S. Pat. No. 5,445,934, U.S. Pat. No. 5,744,305 and U.S. Pat. No. 5,807,522. The main characteristic of the solid support should be to conserve the hybridization characteristics of the capture probes on the target nucleotide fragments while at the same time generating a minimum background noise for the method of detection.

Three main types of fabrication can be distinguished for immobilizing the probes on the support.

First of all, there is a first technique which consists in depositing presynthesized probes. The attachment of the probes is carried out by direct transfer, by means of micropipettes or of microdots or by means of an inkjet device. This technique allows the attachment of probes having a size ranging from a few bases (5 to 10) up to relatively large sizes of 60 bases (printing) to a few hundred bases (microdeposition): Printing is an adaptation of the method used by inkjet printers. It is based on the propulsion of very small spheres of fluid (volume<1 nl) at a rate that may reach 4000 drops/second. The printing does not involve any contact between the system releasing the fluid and the surface on which it is deposited.

Microdeposition consists in attaching long probes of a few tens to several hundred bases to the surface of a glass slide. These probes are generally extracted from databases and are in the form of amplified and purified products. This technique makes it possible to produce chips called microarrays that carry approximately ten thousand spots, called recognition zones, of DNA on a surface area of a little less than 4 cm². The use of nylon membranes, referred to as “macroarrays”, which carry products that have been amplified, generally by PCR, with a diameter of 0.5 to 1 mm and the maximum density of which is 25 spots/cm², should not however be forgotten. This very flexible technique is used by many laboratories. In the present invention, the latter technique is considered to be included among biochips. A certain volume of sample can, however, be deposited at the bottom of a microtitration plate, in each well, as in the case in patent applications WO-A-00/71750 and FR 00/14896, or a certain number of drops that are separate from one another can be deposited at the bottom of one and the same Petri dish, according to another patent application, FR00/14691.

The second technique for attaching the probes to the support or chip is called in situ synthesis. This technique results in the production of short probes directly at the surface of the chip. It is based on in situ oligonucleotide synthesis (see, in particular, patent applications WO 89/10977 and WO 90/03382), and is based on the oligonucleotide synthesizer process. It consists in moving a reaction chamber, in which the oligonucleotide extension reaction takes place, along the glass surface.

Finally, the third technique is called photolithography, which is a process that is responsible for the biochips developed by Affymetrix. It is also an in situ synthesis. Photolithography is derived from microprocessor techniques. The surface of the chip is modified by the attachment of photolabile chemical groups that can be light-activated. Once illuminated, these groups are capable of reacting with the 3′ end of an oligonucleotide. By protecting this surface with masks of defined shapes, it is possible to selectively illuminate and therefore activate areas of the chip where it is desired to attach one or other of the four nucleotides. The successive use of various masks makes it possible to alternate cycles of protection/reaction and therefore to produce the oligonucleotide probes on spots of approximately a few tens of square micrometers (μm²). This resolution makes it possible to create up to several hundred thousand spots on a surface area of a few square centimeters (cm²). Photolithography has advantages: in bulk in parallel, it makes it possible to create a chip of N-mers in only 4×N cycles. All these techniques can be used with the present invention.

For the purpose of the present invention, the determination of the expression (step c) of the method according to the invention) of a target gene can be carried out by any of the protocols known to those skilled in the art.

In general, the expression of a target gene can be analyzed by detection of the mRNAs (messenger RNAs) which are transcribed from the target gene at a given instant or by the detection of the proteins derived from these mRNAs.

When the specific reagent is an amplification primer, the expression of a target gene can be determined in the following way:

1) after having extracted, as biological material, the total RNA (comprising the transfer RNAs (tRNAs), the ribosomal RNAs (rRNAs) and the messenger RNAs (mRNAs)) from a biological sample as presented above, a reverse transcription step is carried out in order to obtain the complementary DNAs (or cDNAs) of said mRNAs. By way of indication, this reverse transcription reaction can be carried out using a reverse transcriptase enzyme which makes it possible to obtain, from an RNA fragment, a complementary DNA fragment. The reverse transcriptase enzyme originating from AMV (avian myoblastosis virus) or from MMLV (moloney murine leukemia virus) can in particular be used. When it is more particularly desired to obtain only the cDNAs of the mRNAs, this reverse transcription step is carried out in the presence of nucleotide fragments comprising only thymine bases (polyT), which hybridize by complementarity on the polyA sequence of the mRNAs so as to form a polyT-polyA complex which then serves as a starting point for the reverse transcription reaction carried out by the reverse transcriptase enzyme. cDNAs complementary to the mRNAs derived from a target gene (target gene-specific cDNA) and cDNAs complementary to the mRNAs derived from genes other than the target gene (cDNAs not specific for the target gene) are then obtained.

2) the amplification primer(s) specific for a target gene is (are) brought into contact with the target gene-specific cDNAs and the cDNAs not specific for the target gene. The amplification primer(s) specific for a target gene hybridize(s) with the target gene-specific cDNAs and a predetermined region, of known length, of the cDNAs originating from the mRNAs derived from the target gene is specifically amplified. The cDNAs not specific for the target gene are not amplified, whereas a large amount of target gene-specific cDNAs is then obtained. For the purpose of the present invention, reference is made, without distinction, to “target gene-specific cDNAs” or to “cDNAs originating from the mRNAs derived from the target gene”. This step can be carried out in particular by means of a PCR-type amplification reaction or by any other amplification technique as defined above. By PCR, it is also possible to simultaneously amplify several different cDNAs, each one being specific for various target genes, by using several pairs of different amplification primers, each one being specific for a target gene: reference is then made to multiplex amplification.

3) the expression of the target gene is determined by detecting and quantifying the target gene-specific cDNAs obtained in step 2) above. This detection can be carried out after electrophoretic migration of the target gene-specific cDNAs according to their size. The gel and the migration medium can include ethydium bromide so as to allow direct detection of the target gene-specific cDNAs when the gel is placed, after a given migration period, on a UV (ultraviolet)-ray light table, through the emission of a light signal. The greater the amount of target gene-specific cDNAs, the brighter this light signal. These electrophoresis techniques are well known to those skilled in the art. The target gene-specific cDNAs can also be detected and quantified using a quantification range obtained by means of an amplification reaction carried out until saturation. In order to take into account the variability and enzymatic efficiency that may be observed during the various steps (reverse transcription, PCR, etc.), the expression of a target gene of various groups of patients can be normalized by simultaneously determining the expression of a “housekeeping” gene, the expression of which is similar in the various groups of patients. By realizing a ratio of the expression of the target gene to the expression of the housekeeping gene, i.e. by realizing a ratio of the amount of target gene-specific cDNAs to the amount of housekeeping gene-specific cDNAs, any variability between the various experiments is thus corrected. Those skilled in the art may refer in particular to the following publications: Bustin S A, J Mol Endocrinol, 2002, 29: 23-39; Giulietti A Methods, 2001, 25: 386-401.

When the specific reagent is a hybridization probe, the expression of a target gene can be determined in the following way:

1) after having extracted, as biological material, the total RNA from a biological sample as presented above, a reverse transcription step is carried out as described above in order to obtain cDNAs complementary to the mRNAs derived from a target gene (target gene-specific cDNA) and cDNAs complementary to the mRNAs derived from genes other than the target gene (cDNA not specific for the target gene).

2) all the cDNAs are brought into contact with a support, on which are immobilized capture probes specific for the target gene whose expression it is desired to analyze, in order to carry out a hybridization reaction between the target gene-specific cDNAs and the capture probes; the cDNAs not specific for the target gene do not hybridize to the capture probes. The hybridization reaction can be carried out on a solid support which includes all the materials as indicated above. According to a preferred embodiment, the hybridization probe is immobilized on a support. Preferably, the support is a biochip. The hybridization reaction can be preceded by a step of enzymatic amplification of the target gene-specific cDNAs as described above, so as to obtain a large amount of target gene-specific cDNAs and to increase the probability of a target gene-specific cDNA hybridizing to a capture probe specific for the target gene. The hybridization reaction may also be preceded by a step for labeling and/or cleaving the target gene-specific cDNAs as described above, for example using a labeled deoxyribonucleotide triphosphate for the amplification reaction. The cleavage can be carried out in particular by the action of imidazole and manganese chloride. The target gene-specific cDNA can also be labeled after the amplification step, for example by hybridizing a labeled probe according to the sandwich hybridization technique described in document WO-A-91/19812. Other preferred specific methods for labeling and/or cleaving nucleic acids are described in applications WO 99/65926, WO 01/44507, WO 01/44506, WO 02/090584 and WO 02/090319.

3) a step for detection of the hybridization reaction is subsequently carried out. The detection can be carried out by bringing the support on which the capture probes specific for the target gene are hybridized with the target gene-specific cDNAs into contact with a “detection” probe labeled with a label, and detecting the signal emitted by the label. When the target gene-specific cDNA has been labeled beforehand with a label, the signal emitted by the label is detected directly.

When the specific reagent is a hybridization probe, the expression of a target gene can also be determined in the following way:

1) after having extracted, as biological material, the total RNA from a biological sample as presented above, a reverse transcription step is carried out as described above in order to obtain the cDNAs of the mRNAs of the biological material. The polymerization of the complementary RNA of the cDNA is subsequently carried out using a T7 polymerase enzyme which functions under the control of a promoter and which makes it possible to obtain, from a DNA template, the complementary RNA. The cRNAs of the cDNAs of the mRNAs specific for the target gene (reference is then made to target gene-specific cRNA) and the cRNAs of the cDNAs of the mRNAs not specific for the target gene are then obtained.

2) all the cRNAs are brought into contact with a support on which are immobilized capture probes specific for the target gene whose expression it is desired to analyze, in order to carry out a hybridization reaction between the target gene-specific cRNAs and the capture probes; the cRNAs not specific for the target gene do not hybridize to the capture probes. When it is desired to simultaneously analyze the expression of several target genes, several different capture probes can be immobilized on the support, each one being specific for a target gene. The hybridization reaction can also be preceded by a step for labeling and/or cleaving the target gene-specific cRNAs as described above.

3) a step for detecting the hybridization reaction is subsequently carried out. The detection can be carried out by bringing the support on which the capture probes specific for the target gene are hybridized with the target gene-specific cRNA into contact with a “detection” probe labeled with a label, and detecting the signal emitted by the label. When the target gene-specific cRNA has been labeled beforehand with a label, the signal emitted by the label is detected directly. The use of cRNA is particularly advantageous when a support of biochip type on which a large number of probes are hybridized is used. According to a specific embodiment of the invention, steps B and C are carried out at the same time. This preferred method can in particular be carried out by “real time NASBA”, which groups together, in a single step, the NASBA amplification technique and real-time detection which uses “molecular beacons”. The NASBA reaction takes place in the tube, producing the single-stranded RNA with which the specific “molecular beacons” can simultaneously hybridize to give a fluorescent signal. The formation of the new RNA molecules is measured in real time by continuous verification of the signal in a fluorescent reader. Unlike an RT-PCR amplification, NASBA amplification can take place with contaminating DNA being present in the sample. It is not therefore necessary to verify that the DNA has indeed been completely eliminated during the RNA extraction.

Surprisingly, the inventors have demonstrated that the analysis of the expression of target genes selected from 74 genes, as presented in table 1 below, is highly relevant for the early diagnosis of breast cancer.

TABLE 1
List of the 74 genes differentially expressed during the development of a
stage I/II breast cancer

| SEQ ID No. | Sequence description | Genbank No. |
|---|---|---|
| 1 | Centrosome-associated protein 350 [CAP350] | NM_014810 |
| 2 | Hypothetical protein MGC23401 | NM_144982 |
| 3 | Trophoblast-derived noncoding RNA [TncRNA] | AF001893 (Hs.523789) |
| 4 | Vacuolar protein sorting 35 (yeast) [PUM2] | NM_015317 |
| 5 | Ribosomal protein L36a-like [RPL36AL] | NM_001001 |
| 6 | Mitochondrial ribosomal protein L51 [MRPL51] | NM_016497 |
| 7 | KIAA0794 protein [KIAA0794] | XM_087353 |
| 8 | Transcribed locus | CA775887 (Hs. 388575) |
| 9 | Hypothetical protein MGC14817 [MGC14817] | NM_032338 |
| 10 | Hypothetical protein FLJ11046 | NM_018309 |
| 11 | Pleckstrin homology, Sec7 and coiled-coil domains 4 [PSCD4] | NM_013385 |
| 12 | Lactate dehydrogenase B [LDHB] | NM_002300 |
| 13 | NADH dehydrogenase (ubiquinone) alpha subcomplex 1 [NDUFA1] | NM_004541 |
| 14 | Muscleblind-like (Drosophila) [MBNL1] | NM_021038 |
| 15 | Ubiquitin specific protease 25 [USP25] | NM_013396 |
| 16 | TATA element modulatory factor 1 [TMF1] | NM_007114 |
| 17 | Ring finger protein 19 [RNF19] | NM_015435 |
| 18 | Signal peptidase complex subunit 3 homolog (S. cerevisiae) [SPCS3] | NM_021928 |
| 19 | Enhancer of polycomb homolog 1 (Drosophila) [EPC1] | NM_025209 |
| 20 | Zinc finger, matrin type 2 [ZMAT2] | NM_144723 |
| 21 | Image clone 3069209 | BF512254 |
| 22 | ORM1-like 3 (S. cerevisiae) [ORMDL3] | NM_139280 |
| 23 | CDNA FLJ11397 fis, clone HEMBA1000622 | AW962458 (Hs. 470871) |
| 24 | Tankyrase, TRF1-interacting ankyrin-related ADP-ribose polymerase [TNKS] | NM_003747 |
| 25 | Ribosomal protein S23 [RPS23] | NM_001025 |
| 26 | CDNA clone IMAGE: 5263531 | AK025902 (Hs.399763) |
| 27 | PABP1-dependent poly A-specific ribonuclease subunit [PAN3] | NM_175854 |
| 28 | Hypothetical protein FLJ21924 | NM_024774 |
| 29 | CDNA FLJ42313 fis, clone TRACH2019425 | AK124306 (Hs.386042) |
| 30 | Family with sequence similarity 49, member B [FAM49B] | NM_016623 |
| 31 | Dicer1, Dcr-1 homolog (Drosophila) [DICER1] | NM_030621 |
| 32 | Ribosomal protein L37 [RPL37] | NM_000997 |
| 33 | UDP-glucose ceramide glucosyltransferase [UGCG] | NM_003358 |
| 34 | Complement component (3b/4b) receptor 1 [CR1] | NM_000573 |
| 35 | KIAA1702 protein | AB051489 (Hs.485628) |
| 36 | Hypothetical protein FLJ10618 | NM_018155 |
| 37 | Hypothetical protein LOC146174 | NM_173501 |
| 38 | MRNA; cDNA DKFZp686D22106 (from clone DKFZp686D22106) | CR933609 (Hs. 445036) |
| 39 | Anterior pharynx defective 1 homolog A (C. elegans) [APH1A] | NM_016022 |
| 40 | U2-associated SR140 protein [SR140] | NM_031553 |
| 41 | Androgen-induced proliferation inhibitor [APRIN] | NM_015032 |
| 42 | Peptidylprolyl isomerase D (cyclophilin D) [PPID] | NM_005038 |
| 43 | Mitochondrial ribosomal protein S17 [MRPS17] | NM_015969 |
| 44 | Adaptor-related protein complex 1, sigma 2 subunit [AP1S2] | NM_003916 |
| 45 | Heat shock 90 kDa protein 1, alpha [HSPCA] | NM_005348 |
| 46 | GNAS complex locus [GNAS] | NM_000516 |
| 47 | 5-azacytidine induced 2 [AZI2] | NM_022461 |
| 48 | BCL2-like 1 [BCL2L1] | NM_001191 |
| 49 | Bobby sox homolog (Drosophila) [BBX] | NM_020235 |
| 50 | Calcium-transporting ATPase, type 2C, member 1 [ATP2C1] | NM_001001485 |
| 51 | Cathepsin Z [CTSZ] | NM_001336 |
| 52 | CDNA FLJ26120 fis, clone SYN00419 | AK129631 (Hs.433995) |
| 53 | COMM domain containing 6 [COMMD6] | NM_203495 |
| 54 | Cytochrome c oxidase subunit VIIb [COX7B] | NM_001866 |
| 55 | Cytoplasmic polyadenylation element binding protein 2 [CPEB2] | NM_182485 |
| 56 | Endoplasmic reticulum-golgi intermediate compartment 32 kDa protein | NM_020462 |
| | [KIAA1181] | |
| 57 | Ewing sarcoma breakpoint region 1 [EWSR1] | NM_005243 |
| 58 | Glyceraldehyde-3-phosphate dehydrogenase [GAPD] | NM_002046 |
| 59 | GRB2-associated binding protein 2 [GAB2] | NM_012296 |
| 60 | Killer cell lectin-like receptor subfamily C, member 1 or 2 [KLRC1/KLRC2] | NM_002259 |
| 61 | Killer cell lectin-like receptor subfamily F1 [KLRF1] | NM_016523 |
| 62 | Metastasis associated lung adenocarcinoma transcript 1 [MALAT1] | BX538238 (Hs.187199) |
| 63 | MRNA; cDNA DKFZp586O0724 | BU676985 (Hs.159115) |
| 64 | Nipped-B homolog (Drosophila) [NIPBL] | NM_015384 |
| 65 | Prader-Willi/Angelman region-1 [PAR1] | BE783065 (Hs.546847) |
| 66 | PRO1550 | AF086013 (Hs.371588) |
| 67 | Protein phosphatase 2, regulatory subunit B (B56), epsilon isoform | NM_006246 |
| | [PPP2R5E] | |
| 68 | RP42 homolog [RP42] | NM_020640 |
| 69 | Special AT-rich sequence binding protein 1 [SATB1] | NM_002971 |
| 70 | Tubulin beta 2 [TUBB2] | NM_001069 |
| 71 | Ubiquitin-fold modifier 1 [Ufm1] | NM_016617 |
| 72 | v-myb myeloblastosis viral oncogene homolog [MYBL1] | XM_034274 |
| 73 | WNK lysine deficient protein kinase 1 [WNK1] | NM_018979 |
| 74 | Zinc finger, MYND domain containing 11 [ZMYND11] | NM_006624 |

Among these genes, it is possible to distinguish genes for which the function is known but which have never been associated with breast cancer (SEQ ID Nos. 3 to 6; 11; 13 to 15; 17; 25; 31 to 34; 39; 42 to 46; 48; 50; 51; 54; 60; 62; 64; 67; 69; 70; 72 to 74) and also genes for which the function is unknown (SEQ ID Nos. 1; 2; 7 to 10; 16; 18 to 23; 26 to 30; 35 to 38; 40; 47; 49; 52; 53; 55 to 57; 61; 63; 65; 66; 68; 71).

All the isoforms of the genes according to the invention are relevant for the present invention. In this respect, it should in particular be noted that several variants exist for the target gene of SEQ ID No. 14; only the first variant is presented in the table above, but the variants having Genbank accession number: NM_{—}207292; NM_{—}207293; NM_{—}207294; NM_{—}207295; NM_{—}207296; NM_{—}207297 are just as relevant for the purpose of the present invention.

Similarly, for SEQ ID No. 17, only the first variant is presented in the table above, but the variant having Genbank accession number NM_{—}183419 is just as relevant for the purpose of the present invention.

Similarly, for SEQ ID No. 31, only the first variant is presented in the table above, but the variant having Genbank accession number NM_{—}177438 is just as relevant for the purpose of the present invention.

Similarly, for SEQ ID No. 34, only the first variant is presented in the table above, but the variant having Genbank accession number NM_{—}000651 is just as relevant for the purpose of the present invention.

Similarly, for SEQ ID No. 41, only the first variant is presented in the table above, but the variant having Genbank accession number NM_{—}015928 is just as relevant for the purpose of the present invention.

For the target gene of SEQ ID No. 46, only the first variant is presented in the table above, but the variants having Genbank accession number: NM_{—}016592; NM_{—}080425; NM_{—}080426 are just as relevant for the purpose of the present invention.

For the target gene of SEQ ID No. 47, only the first variant is presented in the table above, but the variant having Genbank accession number NM_{—}203326 is just as relevant for the purpose of the present invention.

For the target gene of SEQ ID No. 48, only the first variant is presented in the table above, but the variant having Genbank accession number NM_{—}138578 is just as relevant for the purpose of the present invention.

For the target gene of SEQ ID No. 50, only the first variant is presented in the table above, but the variants having Genbank accession number NM_{—}001001485; NM_{—}001001486; NM_{—}001001487; NM_{—}014382 are just as relevant for the purpose of the present invention.

For the target gene of SEQ ID No. 53, only the first variant is presented in the table above, but the variant having Genbank accession number NM_{—}203497 is just as relevant for the purpose of the present invention.

For the target gene of SEQ ID No. 55, only the first variant is presented in the table above, but the variant having Genbank accession number NM_{—}182646 is just as relevant for the purpose of the present invention.

For the target gene of SEQ ID No. 57, only the first variant is presented in the table above, but the variant having Genbank accession number Genbank NM_{—}013986 is just as relevant for the purpose of the present invention.

For the target gene of SEQ ID No. 59, only the first variant is presented in the table above, but the variant having Genbank accession number NM_{—}080491 is just as relevant for the purpose of the present invention.

For the target gene of SEQ ID No. 60, only the first variant is presented in the table above, but the variants having Genbank accession number: NM_{—}002259; NM_{—}002260; NM_{—}007328; NM_{—}213657; NM_{—}213658 are just as relevant for the purpose of the present invention.

For the target gene of SEQ ID No. 64, only the first variant is presented in the table above, but the variant having Genbank accession number NM_{—}133433 is just as relevant for the purpose of the present invention.

For the target gene of SEQ ID No. 74, only the first variant is presented in the table above, but the variant having Genbank accession number NM_{—}212479 is just as relevant for the purpose of the present invention.

Furthermore, the inventors have demonstrated that the analysis of target genes selected from the 95 genes presented in table 2 below are highly relevant for the diagnosis of advanced breast cancer.

TABLE 2
List of the 95 genes differentially expressed during the development of a
stage III/IV breast cancer

| SEQ ID No. | Sequence description | Genbank No. |
|---|---|---|
| 1 | Centrosome-associated protein 350 [CAP350] | NM_014810 |
| 2 | Hypothetical protein MGC23401 | NM_144982 |
| 3 | Trophoblast-derived noncoding RNA [TncRNA] | AF001893 (Hs.523789) |
| 4 | Vacuolar protein sorting 35 (yeast) [PUM2] | NM_015317 |
| 5 | Ribosomal protein L36a-like [RPL36AL] | NM_001001 |
| 6 | Mitochondrial ribosomal protein L51 [MRPL51] | NM_016497 |
| 13 | NADH dehydrogenase (ubiquinone) alpha subcomplex 1 [NDUFA1] | NM_004541 |
| 14 | Muscleblind-like (Drosophila) [MBNL1] | NM_021038 |
| 20 | Zinc finger, matrin type 2 [ZMAT2] | NM_144723 |
| 26 | CDNA clone IMAGE: 5263531, partial cds | AK025902 (Hs.399763) |
| 28 | Hypothetical protein FLJ21924 | NM_024774 |
| 36 | Hypothetical protein FLJ10618 | NM_018155 |
| 37 | Hypothetical protein LOC283666 | BC048264 (Hs.512943) |
| 39 | Anterior pharynx defective 1 homolog A (C. elegans) [APH1A] | NM_016022 |
| 40 | U2-associated SR140 protein [SR140] | XM_031553 |
| 41 | Androgen-induced proliferation inhibitor [APRIN] | NM_015032 |
| 46 | GNAS complex locus [GNAS] | NM_000516 |
| 48 | BCL2-like 1 [BCL2L1] | NM_001191 |
| 59 | GRB2-associated binding protein 2 [GAB2] | NM_012296 |
| 60 | Killer cell lectin-like receptor subfamily C, member 1 or 2 | NM_002259 |
| | [KLRC1/KLRC2] | |
| 61 | Killer cell lectin-like receptor subfamily F1 [KLRF1] | NM_016523 |
| 63 | mRNA; cDNA DKFZp586O0724 (from clone DKFZp586O0724) | BU676985 (Hs.159115) |
| 65 | Prader-Willi/Angelman region-1 [PAR1] | BE783065 (Hs.546847) |
| 67 | Protein phosphatase 2, regulatory subunit B (B56), epsilon isoform | NM_006246 |
| | [PPP2R5E] | |
| 69 | Special AT-rich sequence binding protein 1 [SATB1] | NM_002971 |
| 70 | Tubulin beta 2 [TUBB2] | NM_001069 |
| 71 | Ubiquitin-fold modifier 1 [Ufm1] | NM_016617 |
| 72 | v-myb myeloblastosis viral oncogene homolog [MYBL1] | XM_034274 |
| 73 | WNK lysine deficient protein kinase 1 [WNK1] | NM_018979 |
| 74 | Zinc finger, MYND domain containing 11 [ZMYND11] | NM_006624 |
| 75 | 30 kDa protein LOC55831 | NM_018447 |
| 76 | ADP-ribosylation factor guanine nucleotide-exchange factor 2 | NM_006420 |
| | [ARFGEF2] | |
| 77 | BTB (POZ) domain containing 5 [BTBD5] | NM_017658 |
| 78 | Cathepsin O [CTSO] | NM_001334 |
| 79 | Centrin, EF-hand protein 2 [CETN2] | NM_004344 |
| 80 | Chromosome 16 open reading frame 35 [C16orf35] | NM_012075 |
| 81 | Chromosome 2 open reading frame 33 [C2orf33] | NM_020194 |
| 82 | Cleavage and polyadenylation specific factor 6, 68 kDa [CPSF6] | NM_007007 |
| 83 | Cysteine-rich motor neuron 1 [CRIM1] | NM_016441 |
| 84 | Enoyl Coenzyme A hydratase domain containing 1 [ECHDC1] | NM_018479 |
| 85 | Erythrocyte membrane protein band 4.2 [EPB42] | NM_000119 |
| 86 | Formin binding protein 3 [FNBP3] | XM_371575 |
| 87 | Hepatitis B virus x associated protein [HBXAP] | NM_016578 |
| 88 | Hypothetical protein HSPC129 | NM_016396 |
| 89 | Hypothetical protein LOC144438 | AK002085 (Hs.92308) |
| 90 | Hypothetical protein MGC33214 | NM_153354 |
| 91 | Hypothetical protein MGC5306 | NM_024116 |
| 92 | Likely ortholog of mouse TORC2-specific protein AVO3 (S. cerevisiae) | NM_152756 |
| | [AVO3] | |
| 93 | Mannosidase, alpha, class 2A, member 1 [MAN2A1] | NM_002372 |
| 94 | Mdm4, p53 binding protein (mouse) [MDM4] | NM_002393 |
| 95 | Nucleobindin 1 [NUCB1] | NM_006184 |
| 96 | Oxysterol binding protein 2 [OSBP2] | NM_001003812 |
| 97 | Phosphoinositide-3-kinase, catalytic, alpha polypeptide [PIK3CA] | NM_006218 |
| 98 | Proteasome (prosome, macropain) inhibitor subunit 1 (PI31) [PSMF1] | NM_006814 |
| 99 | Protein tyrosine phosphatase type IVA, member 2 [PTP4A2] | NM_003479 |
| 100 | Rhesus blood group, D antigen [RHD] | NM_016124 |
| 101 | Ring finger protein 123 [RNF123] | NM_022064 |
| 102 | SH2 domain-containing molecule EAT2 [EAT2] | NM_053282 |
| 103 | Source of immunodominant MHC-associated peptides [SIMP] | NM_178862 |
| 104 | Split hand/foot malformation (ectrodactyly) type 1 [SHFM1] | NM_006304 |
| 105 | Thyroid hormone receptor associated protein 1 [THRAP1] | NM_005121 |
| 106 | Thyroid hormone receptor interactor 12 [TRIP12] | NM_004238 |
| 107 | Transcribed locus | AL037805 (Hs. 445247) |
| 108 | Transducin (beta)-like 1X-linked receptor 1 [TBL1XR1] | NM_024665 |
| 109 | Tubulin, beta 3 [TUBB3] | NM_006086 |
| 110 | Ubiquitination factor E4A (UFD2 homolog, yeast) [UBE4A] | NM_004788 |
| 111 | Zinc finger protein 148 (pHZ-52) [ZNF148] | NM_021964 |
| 112 | 3-alpha hydroxysteroid dehydrogenase, type II [AKR1C3] | NM_003739 |
| 113 | A kinase (PRKA) anchor protein 7 [AKAP7] | NM_004842 |
| 114 | Aminolevulinate, delta-, synthase 2 [ALAS2] | NM_000032 |
| 115 | Ankyrin 1, erythrocytic [ANK1] | NM_000037 |
| 116 | B double prime 1, subunit of RNA polymerase III transcription | NM_018429 |
| | initiation factor IIIB [BDP1] | |
| 117 | Carbonic anhydrase I [CA1] | NM_001738 |
| 118 | Chromosome 19 open reading frame 2 [C19orf2] | NM_003796 |
| 119 | DKFZP564F0522 protein | NM_015475 |
| 120 | Erythrocyte membrane protein band 4.9 (dematin) [EPB49] | NM_001978 |
| 121 | Family with sequence similarity 46, member C [FAM46C] | NM_017709 |
| 122 | guanosine monophosphate reductase [GMPR] | NM_006877 |
| 123 | Homo sapiens, clone IMAGE: 5267398, mRNA cDNA | BX538337 (Hs.40289) |
| | DKFZp686I23208 | |
| 124 | Image clone 3481554 | BF062399 |
| 125 | IMAGE clone 5259272 | BC032890 (Hs.184430) |
| 126 | Integrin, alpha 2b [ITGA2B] | NM_000419 |
| 127 | Interleukin 8 [IL8] | NM_000584 |
| 128 | Leucine rich repeat neuronal 3 [LRRN3] | NM_018334 |
| 129 | Leukocyte receptor cluster (LRC) member 10 [LENG10] | AF211977 |
| 130 | Major histocompatibility complex, class II, DQ alpha 1 [HLA-DQA1] | NM_002122 |
| 131 | Phosphatidylinositol glycan, class K [PIGK] | NM_005482 |
| 132 | Selenium binding protein 1 [SELENBP1] | NM_003944 |
| 133 | SM-11044 binding protein [SMBP] | NM_020123 |
| 134 | Solute carrier family 6 (neurotransmitter transporter, creatine), member | NM_005629 |
| | 8 [SLC6A8] | |
| 135 | TBC1 domain family, member 4 [TBC1D4] | NM_014832 |
| 136 | Tensin [TNS] | NM_022648 |
| 137 | TIA1 cytotoxic granule-associated RNA binding protein [TIA1] | NM_022037 |
| 138 | Transcribed locus | AA456099 (Hs.176376) |
| 139 | Tripartite motif-containing 58 [TRIM58] | NM_015431 |

Among these genes, it is possible to distinguish genes for which the function is known but which have never been associated with breast cancer (SEQ ID Nos. 3 to 6; 13; 14; 39; 46; 48; 60; 67; 69; 70; 72 to 74; 76; 78; 79; 82; 83; 85; 87; 92 to 94; 97; 99 to 101; 103; 105; 110; 111; 113 to 116; 118; 120; 122; 126; 130 to 132; 134; 136; 137) and also genes for which the function is unknown (SEQ ID Nos. 1; 2; 20; 26; 28; 36; 37; 40; 61; 63; 65; 71; 75; 77; 80; 81; 84; 86; 88 to 91; 95; 96; 102; 104; 106 to 109; 119; 121; 123 to 125; 128; 129; 133; 135; 138; 139).

All the isoforms of the genes according to the invention are relevant for the present invention. In this respect, it should in particular be noted that several variants exist for the target gene of SEQ ID No. 14; only the first variant is presented in the table above, but the variants having Genbank accession number: NM_{—}207292; NM_{—}207293; NM_{—}207294; NM_{—}207295; NM_{—}207296; NM_{—}207297 are just as relevant for the purpose of the present invention.

Similarly, for the target gene of SEQ ID No. 41, only the first variant is presented in the table above, but the variant having Genbank accession number NM_{—}015928 is just as relevant for the purpose of the present invention.

Similarly, for the target gene of SEQ ID No. 74, only the first variant is presented in the table above, but the variant having Genbank accession number NM_{—}212479 is just as relevant for the purpose of the present invention.

Similarly, for the target gene of SEQ ID No. 96, only the first variant is presented in the table above, but the variant having Genbank accession number NM_{—}030758 is just as relevant for the purpose of the present invention.

For the target gene of SEQ ID No. 98, only the first variant is presented in the table above, but the variants having Genbank accession number: NM_{—}178578; NM_{—}178579 are just as relevant for the purpose of the present invention.

For the target gene of SEQ ID No. 99, only the first variant is presented in the table above, but the variants having Genbank accession number: NM_{—}080391; NM_{—}080392 are just as relevant for the purpose of the present invention.

Similarly, for the target gene of SEQ ID No. 100, only the first variant is presented in the table above, but the variant having Genbank accession number NM_{—}016225 is just as relevant for the purpose of the present invention.

For the target gene of SEQ ID No. 112, only the first variant is presented in the table above, but the variants having Genbank accession number: NM_{—}016377; NM_{—}138633 are just as relevant for the purpose of the present invention.

For the target gene of SEQ ID No. 115, only the first variant is presented in the table above, but the variants having Genbank accession number: NM_{—}020475; NM_{—}020476; NM_{—}020477; NM_{—}020478; NM_{—}020479; NM_{—}020480; NM_{—}020481 are just as relevant for the purpose of the present invention.

Similarly, for the target gene of SEQ ID No. 137, only the first variant is presented in the table above, but the variant having Genbank accession number NM_{—}022173 is just as relevant for the present invention.

In this respect, the invention relates to a method for the in vitro diagnosis of breast cancer in a patient who may be suffering from a breast cancer, characterized in that it comprises the following steps:
a. biological material is extracted from a biological sample taken from the patient,
b. the biological material is brought into contact with at least one specific reagent chosen from the specific reagents for the target genes with a nucleic sequence having any one of SEQ ID Nos. 1 to 74,
c. the expression of said target genes is determined.

The analysis of the expression of a target gene chosen from any one of SEQ ID Nos. 1 to 74 then makes it possible to have a tool for the diagnosis of breast cancer, and is very suitable for an early diagnosis. It is, for example, possible to analyze the expression of a target gene in a patient for whom the diagnosis is not known, and to compare with known average expression values for the target gene of normal patients and known average expression values for the target gene of patients suffering from an early-stage breast cancer.

According to a specific embodiment of the invention, in step b), the biological material is brought into contact with at least 5, at least 10, at least 15, at least 20, at least 25, at least 30, at least 35, at least 40, at least 45, at least 50, at least 55, at least 60, at least 65, at least 70 or at least 74 specific reagents chosen from the specific reagents for the target genes with a nucleic sequence having any one of SEQ ID NOs. 1 to 74, and, in step c, the expression of at least 5, at least 10, at least 15, at least 20, at least 25, at least 30, at least 35, at least 40, at least 45, at least 50, at least 55, at least 60, at least 65, at least 70 or at least 74 of said genes is determined.

According to a specific embodiment of the invention, the invention relates to an in vitro method for the diagnosis, preferably early diagnosis, of breast cancer in a patient who may be suffering from a breast cancer, characterized in that it comprises the following steps:

a. biological material is extracted from a biological sample taken from the patient,
b. the biological material is brought into contact with at least 46 specific reagents chosen from the specific reagents for the target genes with a nucleic sequence having any one of SEQ ID Nos. 1 to 46,
c. the expression of said target genes is determined.

According to another preferred embodiment of the invention, in step b), the biological material is brought into contact with at least 23 or with 23 specific reagents chosen from the specific reagents for the target genes with a nucleic sequence having any one of SEQ ID Nos. 1 to 23, and, in step c, the expression of at least 23 or 23 of said target genes is determined.

According to a particularly preferred embodiment of the invention, the invention relates to an in vitro method for the diagnosis, preferably early diagnosis, of breast cancer in a patient who may be suffering from a breast cancer, characterized in that it comprises the following steps:

a. biological material is extracted from a biological sample taken from the patient,
b. the biological material is brought into contact with at least 23 specific reagents chosen from the specific reagents for the target genes with a nucleic sequence having any one of SEQ ID Nos. 1 to 23,
c. the expression of said target genes is determined.

According to another preferred embodiment of the invention, in step b), the biological material is brought into contact with at least 8 or with 8 specific reagents chosen from the specific reagents for the target genes with a nucleic sequence having any one of SEQ ID Nos. 1 to 8, and, in step c, the expression of at least 8 or 8 of said genes is determined. According to a preferred embodiment of the invention, the invention relates to an in vitro method for the diagnosis, preferably early diagnosis, of breast cancer in a patient who may be suffering from a breast cancer, characterized in that it comprises the following steps:

a. biological material is extracted from a biological sample taken from the patient,
b. the biological material is brought into contact with at least 8 specific reagents chosen from the specific reagents for the target genes with a nucleic sequence having any one of SEQ ID Nos. 1 to 8,
c. the expression of said target genes is determined.

The use of a restricted panel of genes is particularly suitable for obtaining a prognostic tool. In fact, the analysis of the expression of about ten genes does not require the custom-made fabrication of DNA chips, and can be carried out directly by PCR or NASBA techniques, or low-density chip techniques, which provides a considerable economic asset and a simplified implementation.

The invention also relates to a method for the in vitro diagnosis of breast cancer in a patient who may be suffering from a breast cancer, characterized in that it comprises the following steps:
a. biological material is extracted from a biological sample taken from the patient,
b. the biological material is brought into contact with at least one specific reagent chosen from the specific reagents for the target genes with a nucleic sequence having any one of SEQ ID Nos. 1 to 6; No. 1 to 6; 13; 14; 20; 26; 28; 36 to 41; 46; 48; 59 to 61; 63; 65; 67; 69 to 139,
c. the expression of said target genes is determined.

The analysis of the expression of a target gene chosen from any one of SEQ ID No. 1 to SEQ ID Nos. 1 to 6; 13; 14; 20; 26; 28; 36 to 41; 46; 48; 59 to 61; 63; 65; 67; 69 to 139 then makes it possible to provide a tool for the diagnosis of breast cancer, which is very suitable for the diagnosis of a late-stage cancer. It is, for example, possible to analyze the expression of a target gene in a patient for whom the diagnosis is not known, and to compare with known average expression values for the target gene of normal patients and known average expression values for the target gene of patients suffering from a late-stage breast cancer. This tool also makes it possible, for example, to monitor a treatment prescribed for a patient suffering from an advanced breast cancer.

According to a specific embodiment of the invention, in step b), the biological material is brought into contact with at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95 or at least 97 specific reagents chosen from the specific reagents for the target genes with a nucleic sequence having any one of SEQ ID Nos. 1 to 6; 13; 14; 20; 26; 28; 36 to 41; 46; 48; 59 to 61; 63; 65; 67; 69 to 139, and, in step c, the expression of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95 or at least 97 of said target genes is determined.

According to a preferred embodiment of the invention, the invention relates to a method for the in vitro late diagnosis of breast cancer in a patient who may be suffering from a breast cancer, characterized in that it comprises the following steps:

a. biological material is extracted from a biological sample taken from the patient,
b. the biological material is brought into contact with at least 54 or 54 specific reagents chosen from the specific reagents for the target genes with a nucleic sequence having any one of SEQ ID Nos. 1 to 6; 13; 14; 20; 26; 28; 38 to 41; 69; 74 to 110,
c. the expression of said target genes is determined.

According to another preferred embodiment of the invention, in step b), the biological material is brought into contact with at least 29 or with 29 specific reagents chosen from the specific reagents for the target genes with a nucleic sequence having any one of SEQ ID Nos. 1; 2; 4 to 6; 13; 14; 20; 26; 38; 39; 41; 69; 75; 79 to 81; 87; 89; 93; 95 to 96; 101; 103 to 106; 108; 110, and, in step c, the expression of at least 29 or 29 of said target genes is determined.

According to a specific embodiment of the invention, the invention relates to a method for the in vitro early diagnosis of breast cancer in a patient who may be suffering from a breast cancer, characterized in that it comprises the following steps:

a. biological material is extracted from a biological sample taken from the patient,
b. the biological material is brought into contact with at least 29 or 29 specific reagents chosen from the specific reagents for the target genes with a nucleic sequence having any one of SEQ ID Nos. SEQ ID Nos 1; 2; 4 to 6; 13; 14; 20; 26; 38; 39; 41; 69; 75; 79 to 81; 87; 89; 93; 95 to 96; 101; 103 to 106; 108; 110,
c. the expression of said target genes is determined.

According to another preferred embodiment of the invention, in step b), the biological material is brought into contact with at least 10 or with 10 specific reagents chosen from the specific reagents for the target genes with a nucleic sequence having any one of SEQ ID Nos. 1; 2; 4; 6; 13; 14; 26; 69; 81; 105, and, in step c, the expression of at least 10 or 10 of said target genes is determined.

According to a preferred embodiment of the invention, the invention relates to an in vitro method for the diagnosis, preferably late diagnosis, of breast cancer in a patient who may be suffering from a breast cancer, characterized in that it comprises the following steps:

a. biological material is extracted from a biological sample taken from the patient,
b. the biological material is brought into contact with at least 10 or 10 specific reagents chosen from the specific reagents for the target genes with a nucleic sequence having any one of SEQ ID Nos. 1; 2; 4; 6; 13; 14; 26; 69; 81; 105,
c. the expression of said target genes is determined.

The use of a restricted panel of genes is particularly suitable for obtaining a prognostic tool. In fact, the analysis of the expression of about ten genes does not require the custom-made fabrication of DNA chips, and can be carried out directly by PCR or NASBA techniques, or low-density chip techniques, which provides a considerable economic asset and a simplified implementation.

Irrespective of the variant of the method according to the invention, the biological sample taken from the patient is preferably a blood sample. This makes it possible to obtain a method of diagnosis that is easy to implement and relatively painless for the patient. Irrespective of the variant of the method according to the invention, the biological material extracted in step a) preferably comprises nucleic acids, which allows an easy and rapid analysis of the expression of the target gene(s) in step c). In this case, said specific reagents of step b) are preferably hybridization probes. These hybridization probes are preferably immobilized on a support, which is preferably a biochip. This biochip then allows the simultaneous analysis of all the target genes according to the invention.

The invention also relates to a support, as defined above, comprising at least 8 specific hybridization probes for target genes with a nucleic sequence having any one of SEQ ID Nos. 1 to 8. The invention also relates to a support, as defined above, consisting of 8 specific hybridization probes for target genes with a nucleic sequence having any one of SEQ ID Nos. 1 to 8.

The invention also relates to a support, as defined above, comprising at least 23 specific hybridization probes for target genes with a nucleic sequence having any one of SEQ ID Nos. 1 to 23. The invention also relates to a support, as defined above, consisting of 23 specific hybridization probes for target genes with a nucleic sequence having any one of SEQ ID Nos. 1 to 23.

The invention also relates to a support, as defined above, comprising at least 46 specific hybridization probes for target genes with a nucleic sequence having any one of SEQ ID Nos. 1 to 46. This support preferably also comprises at least 28 specific hybridization probes for target genes with a nucleic sequence having any one of SEQ ID Nos. 47 to 74. The invention also relates to a support, as defined above, consisting of 46 specific hybridization probes for target genes with a nucleic sequence having any one of SEQ ID Nos. 1 to 46. This support preferably also consists of 28 specific hybridization probes for target genes with a nucleic sequence having any one of SEQ ID Nos. 47 to 74.

The invention also relates to the use of a support as defined above, for the early diagnosis of a breast cancer. The invention also relates to a kit for the early diagnosis of a breast cancer, comprising a support as defined above.

The invention also relates to a support comprising at least or consisting of 10 specific hybridization probes for target genes with a nucleic sequence having any one of SEQ ID Nos. 1; 2; 4; 6; 13; 14; 26; 69; 81; 105.

The invention also relates to a support comprising at least or consisting of 29 specific hybridization probes for target genes with a nucleic sequence having any one of SEQ ID Nos. 1; 2; 4 to 6; 13; 14; 20; 26; 38; 39; 41; 69; 75; 79 to 81; 87; 89; 93; 95 to 96; 101; 103 to 106; 108; 110.

The invention also relates to a support comprising at least or consisting of 54 specific hybridization probes for target genes with a nucleic sequence having any one of SEQ ID Nos. 1 to 6; 13; 14; 20; 26; 28; 38 to 41; 69; 74 to 111. This support preferably also comprises specific hybridization probes for target genes with a nucleic sequence having any one of SEQ ID Nos. 36; 37; 46; 48; 59 to 61; 63; 65; 67; 70 to 72; 73; 112 to 139. The invention also relates to the use of a support as defined above, for the late diagnosis of a breast cancer.

Finally, the invention relates to a kit for the early diagnosis of a breast cancer, comprising a support as defined above.

The attached figures are given by way of explanatory examples and are in no way limiting in nature. They will make it possible to understand the invention more clearly.

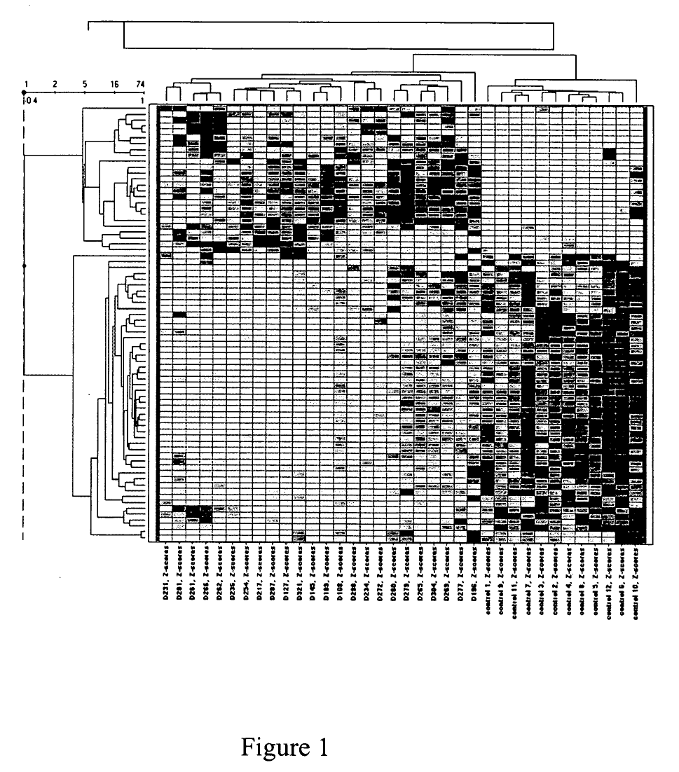

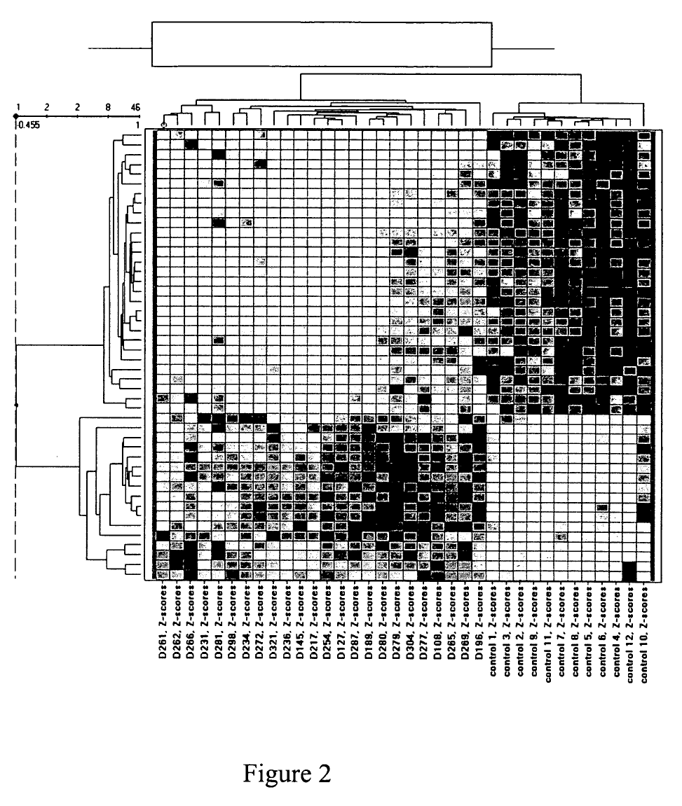

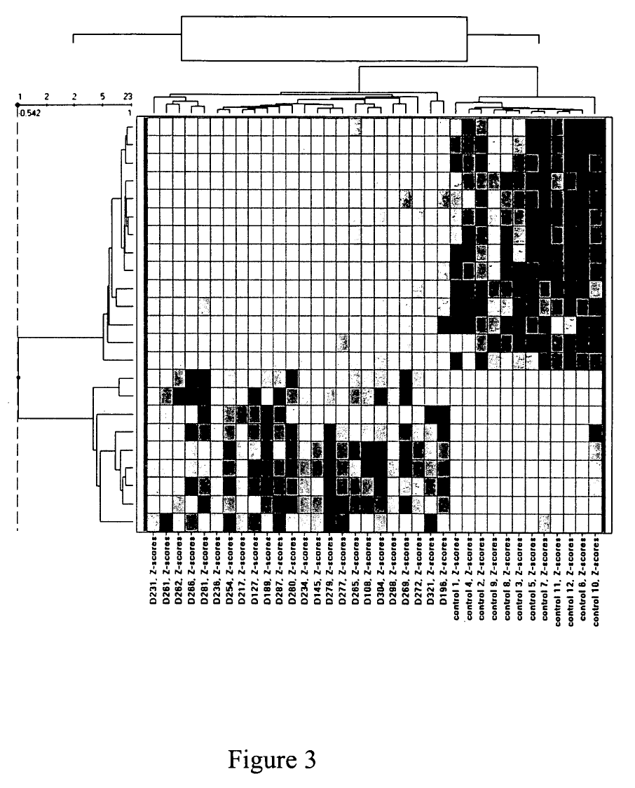

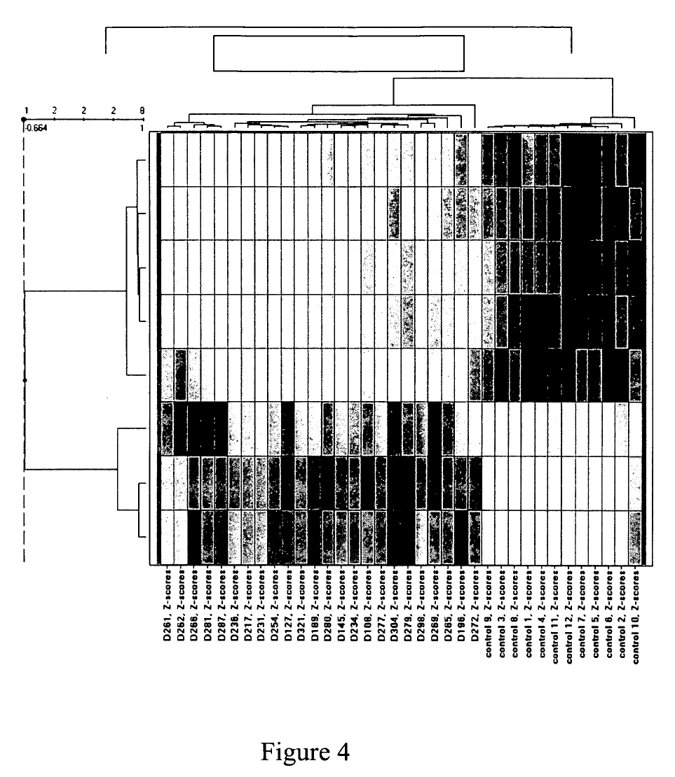

FIGS. 1 to 4 represent the analysis of hierarchical clustering of blood samples obtained from 24 patients suffering from an early-stage cancer (C I/II, also called D) and 12 control patients (normal donors), using the expression of 74 (FIG. 1), 46 (FIG. 2), 23 (FIG. 3) or 8 (FIG. 4) genes identified by algorithmic analysis. The hierarchical clustering function of the Spotfire software organizes the C I/II and control patients in columns, and the genes in rows so as to obtain in adjacent position the patients or the genes with comparable expression profiles. Pearson's correlation coefficient was used as a similarity index for the genes and the patients. The results correspond to the affymetrix fluorescence level normalized with the “bioconductor” tool. In order to take into account the constitutive differences in expression between the genes, the levels of expression of each gene were normalized by calculating a reduced centered variable. The white represents the low levels of expression, the gray the intermediate levels and the black the high levels. The height of the branches of the dendrogram indicates the index of similarity between the expression profiles.

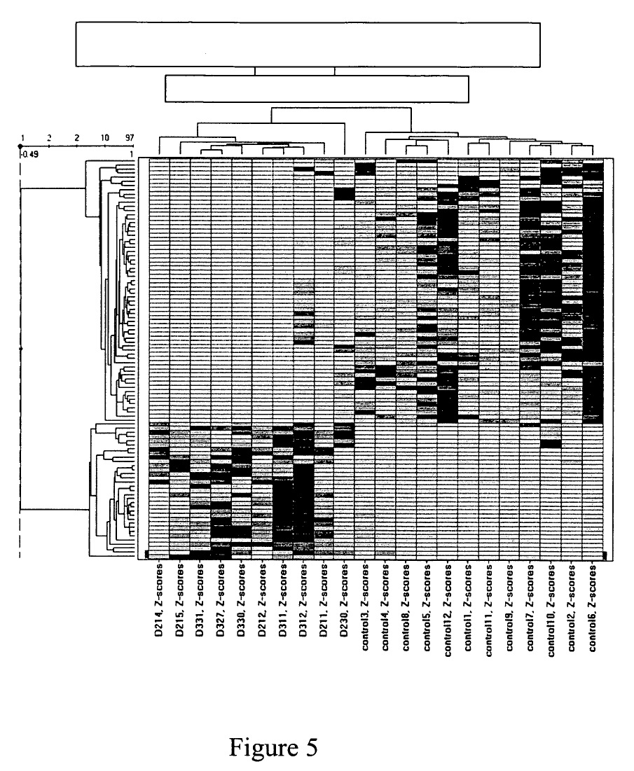

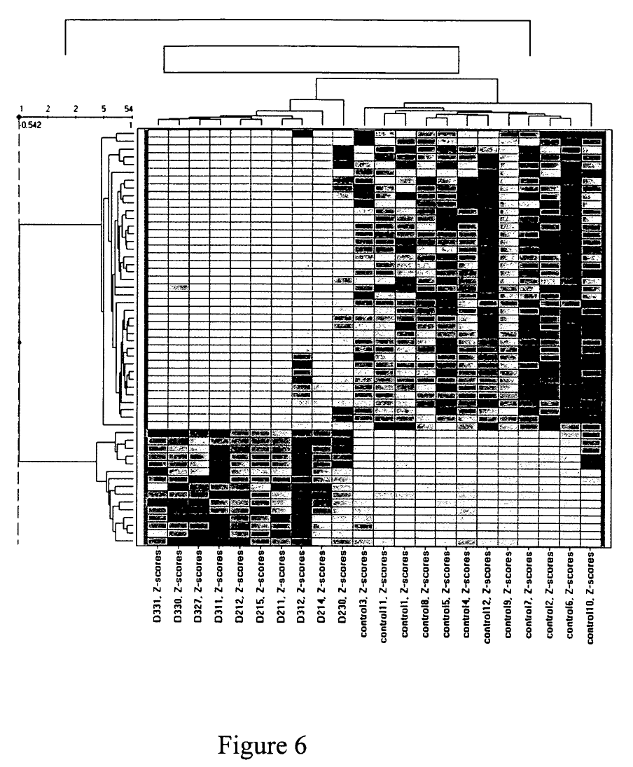

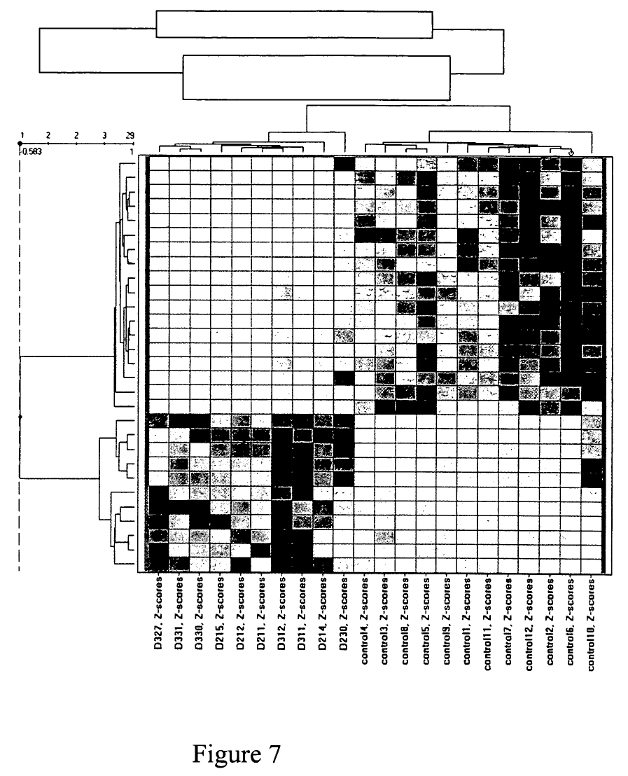

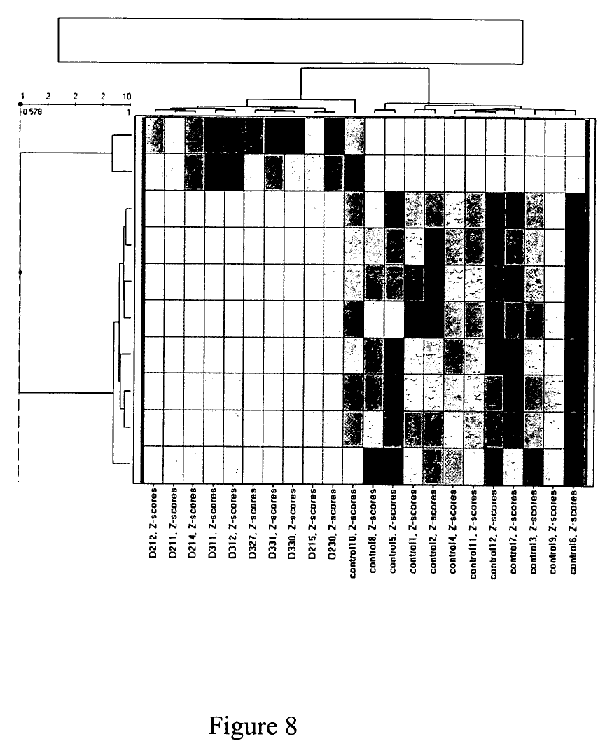

FIGS. 5 to 8 represent the analysis of hierarchical clustering of blood samples obtained from 10 patients suffering from an advanced-stage cancer (C II/IV, referenced D) and 12 control patients (normal donors) using the expression of 97 (FIG. 5), 54 (FIG. 6), 29 (FIG. 7), or 10 (FIG. 8) genes identified by algorithmic analysis. The hierarchical clustering function of the Spotfire software organizes the C III/IV and control patients in columns, and the genes in rows so as to obtain in adjacent position the patients or the genes with comparable expression profiles. Pearson's correlation coefficient was used as a similarity index for the genes and the patients. The results correspond to the affymetrix fluorescence level normalized with the “bioconductor” tool. In order to take into account the constitutive differences in expression between the genes, the levels of expression of each gene were normalized by calculating a reduced centered variable. The white represents the low levels of expression, the gray the intermediate levels and the black the high levels. The height of the branches of the dendrogram indicates the index of similarity between the expression profiles.

The following examples are given by way of illustration and are in no way limiting in nature. They will make it possible to understand the invention more clearly.

### EXAMPLE 1

### Demonstration of an Expression Profile for Breast Cancer Diagnosis Using a Blood Sample

Biological sample characteristics: The example presented hereinafter was carried out using 46 blood samples (5 ml of whole blood, taken in two PaxGene tubes). These samples included 12 blood samples originating from normal control patients (S, obtained from the French Blood Bank) and 24 samples from patients suffering from a phase I/II breast cancer (Ci/ii).

Extraction of the biological material (total RNA) from the biological sample: The blood samples were collected directly in PAXGene™ Blood RNA tubes (PreAnalytix, Frankin Lakes, USA). After the step in which the blood sample was taken, and in order to obtain complete lysis of the cells, the tubes were left at ambient temperature for 4 h and then stored at −20° C. until extraction of the biological material. More specifically, in this protocol, the total RNA was extracted using the PAXGene Blood RNA® kits (PreAnalytix), according to the manufacturer's recommendations. Briefly, the tubes were centrifuged (10 min, 3000 g) in order to obtain a nucleic acid pellet. This pellet was washed and taken up in a buffer containing proteinase K, required for digestion of the proteins (10 min at 55° C.). A further centrifugation (5 min, 19 000 g) was carried out in order to remove the cell debris, and ethanol was added in order to optimize the conditions for binding of the nucleic acids. The total RNA was specifically bound to PAXgene RNA spin columns and, before the elution of said RNA, the contaminating DNA was digested using the RNAse free DNAse set (Qiagen Ltd, Crawley, UK). The quality of the total RNA was analyzed using the AGILENT 2100 bioanalyzer (Agilent Technologies, Waldbronn, Germany). The total RNA comprises the transfer RNAs, the messenger RNAs (mRNAs) and the ribosomal RNAs.

cDNA synthesis, cRNA production and cRNA labeling, and quantification: In order to analyze the expression of the target genes according to the invention, the complementary DNAs (cDNAs) of the mRNAs contained in the total RNA as purified above were obtained from 10 μg of total RNA using 400 units of the SuperScriptII reverse transcription enzyme (Invitrogen) and 100 μmol of poly-T primer containing the T7 promoter (T7-oligo(dT)₂₄-primer, Proligo, Paris, France).

The cDNAs thus obtained were subsequently extracted with phenol/chloroform and precipitated as described previously with ammonium acetate and ethanol, and redissolved in 24 μl of DEPC water. A volume of 20 μl of this purified cDNA solution was subsequently subjected to in vitro transcription using a T7 RNA polymerase which specifically recognizes the T7 polymerase promoter as mentioned above. This transcription makes it possible to obtain the cRNA of the cDNA. This transcription was carried out using an IVT labeling kit (Affymetrix, Santa Clara, Calif.), which makes it possible not only to obtain the cRNA, but also the incorporation of biotinylated pseudouridine bases during the synthesis of the cRNA. The purified cRNAs were subsequently quantified by spectrophotometry, and the cRNA solution was adjusted to a concentration of 1 μg/μl of cRNA. The step consisting of cleavage of these cRNAs was subsequently carried out at 94° C. for 35 min, using a fragmentation buffer (40 mM of tris acetate, pH 8.1, 100 mM of potassium acetate, 30 mM of magnesium acetate) in order to bring about the hydrolysis of the cRNAs and to obtain fragments of 35 to 200 bp. The success of such a fragmentation was verified by means of a 1.5% agarose gel electrophoresis.

Demonstration of an Expression Profile for the Genes which Makes it Possible to Distinguish Between the Control Patients (S) and the Patients Suffering from a Stage I/II Cancer

The expression of approximately 30 000 genes was analyzed and compared between S and C I/II patients. For this, 10 μg of fragmented cRNAs derived from each sample were added to a hybridization buffer (Affymetrix) and 200 μl of this solution were brought into contact for 16 h at 45° C. on an expression chip (Human Genome U133Plus2 GeneChip® (Affymetrix)), which comprises 54 000 groups of probes representing approximately 30 000 genes, according to the Affymetrix protocol.

In order to record the best hybridization and washing performance levels, RNAs described as “control” RNAs that were biotinylated (bioB, bioC, bioD and cre) and oligonucleotides (oligo B2) were also included in the hybridization buffer. After the hybridization step, the biotinylated cRNAs hybridized on the chip were visualized using a solution of streptavidin-phycoerythrin and the signal was amplified using an anti-streptavidin antibody. The hybridization was carried out in a “GeneChip Hybridisation oven” (Affymetrix), and the Euk GE-WS2 protocol of the Affymetrix protocol was followed. The washing and visualization steps were carried out on a “Fluidics Station 450” (Affymetrix). Each U133—Plus_{—}2 chip was subsequently analyzed on an Agilent G3000 GeneArray Scanner at a resolution of 1.5 microns in order to pinpoint the areas hybridized on the chip. This scanner makes it possible to detect the signal emitted by the fluorescent molecules after excitation with an argon laser using the epifluorescence microscope technique. A signal proportional to the amount of cRNAs bound is thus obtained for each position. The signal was subsequently analyzed using the GeneChip Operating Software (GCOS1.2, Affymetrix).

In order to prevent the variations obtained by using various chips, a normalization approach was carried out using the “bioconductor” tool, which makes it possible to harmonize the mean distribution of the raw data for each chip. The results obtained on a chip can then be compared with the results obtained on another chip. The GCOS 1.2 software also made it possible to include a statistical algorithm for deciding whether or not a gene was expressed. Each gene represented on the U133_Plus_{—}2 chip was covered by 11 to 16 pairs of probes of 25 nucleotides. The term “pair of probes” is intended to mean a first probe which hybridizes perfectly (reference is then made to PM or perfect match probes) with one of the cRNAs derived from a target gene, and a second probe, identical to the first probe with the exception of a mismatch (reference is then made to MM or mismatched probe) at the center of the probe. Each MM probe was used to estimate the background noise corresponding to a hybridization between two nucleotide fragments of noncomplementary sequence (Affymetrix technical note “Statistical Algorithms Reference Guide”; Lipshutz, et al., (1999) Nat. Genet. 1 Suppl., 20-24). The remaining 46 samples showed an average of 42.1% of expressed genes.

On the basis of the 54 000 groups of probes, representing approximately 30 000 genes, of the chip, the inventors selected the relevant genes which were correlated with the development of a breast cancer.

The genes which have an expression level that is too low on the majority of the chips and also the genes which do not show any substantial variation between the various chips were excluded (Li et al., 2001, Bioinformatics, 17: 1131-1142). The search for a panel of genes for distinguishing the groups of patients EFS [French Blood Bank] and CI/II was carried out by a Data Mining technique (http://ligarto.org/rdiaz/Papers/jomadas.bioinfo.randomForest.pdf).

This analysis made it possible to reveal a first panel of genes, comprising 46 relevant genes according to the invention (SEQ ID Nos. 1 to 46). A complementary analysis (SAM, significance analysis of microarrays) also made it possible to reveal additional genes which also proved to be very relevant (SEQ ID Nos. 47 to 74).

The increase or decrease in expression of each of these genes, observed in the S patients compared with the C I/II patients, is shown in table 3.

TABLE 3
List of the 74 genes differentially expressed during the development of a breast
cancer

| SEQ | | | C I/II vs. |
|---|---|---|---|
| ID | | | normal |
| No. | Sequence description | Genbank No. | patients |
| 1 | Centrosome-associated protein 350 [CAP350] | NM_014810 | 0.6 |
| 2 | Hypothetical protein MGC23401 | NM_144982 | 0.6 |
| 3 | Trophoblast-derived noncoding RNA [TncRNA] | AF001893 (Hs.523789) | 0.4 |
| 4 | Vacuolar protein sorting 35 (yeast) [PUM2] | NM_015317 | 0.6 |
| 5 | Ribosomal protein L36a-like [RPL36AL] | NM_001001 | 2.8 |
| 6 | Mitochondrial ribosomal protein L51 [MRPL51] | NM_016497 | 2.1 |
| 7 | KIAA0794 protein [KIAA0794] | XM_087353 | 1.7 |
| 8 | Transcribed locus | CA775887 (Hs. 388575) | 0.6 |
| 9 | Hypothetical protein MGC14817 [MGC14817] | NM_032338 | 2.1 |
| 10 | Hypothetical protein FLJ11046 | NM_018309 | 0.7 |
| 11 | Pleckstrin homology, Sec7 and coiled-coil domains 4 [PSCD4] | NM_013385 | 1.5 |
| 12 | Lactate dehydrogenase B [LDHB] | NM_002300 | 2.3 |
| 13 | NADH dehydrogenase (ubiquinone) alpha subcomplex 1 [NDUFA1] | NM_004541 | 1.7 |
| 14 | Muscleblind-like (Drosophila) [MBNL1] | NM_021038 | 0.5 |
| 15 | Ubiquitin specific protease 25 [USP25] | NM_013396 | 0.6 |
| 16 | TATA element modulatory factor 1 [TMF1] | NM_007114 | 0.7 |
| 17 | Ring finger protein 19 [RNF19] | NM_015435 | 0.6 |
| 18 | Signal peptidase complex subunit 3 homolog (S. cerevisiae) [SPCS3] | NM_021928 | 0.6 |
| 19 | Enhancer of polycomb homolog 1 (Drosophila) [EPC1] | NM_025209 | 0.6 |
| 20 | Zinc finger, matrin type 2 [ZMAT2] | NM_144723 | 1.7 |
| 21 | Image clone 3069209 | BF512254 | 0.6 |
| 22 | ORM1-like 3 (S. cerevisiae) [ORMDL3] | NM_139280 | 1.5 |
| 23 | CDNA FLJ11397 fis, clone HEMBA1000622 | AW962458 (Hs. 470871) | 0.6 |
| 24 | Tankyrase, TRF1-interacting ankyrin-related ADP-ribose polymerase | NM_003747 | 0.6 |
| | [TNKS] | | |
| 25 | Ribosomal protein S23 [RPS23] | NM_001025 | 1.8 |
| 26 | CDNA clone IMAGE: 5263531 | AK025902 (Hs.399763) | 0.7 |
| 27 | PABP1-dependent poly A-specific ribonuclease subunit [PAN3] | NM_175854 | 0.6 |
| 28 | Hypothetical protein FLJ21924 | NM_024774 | 0.6 |
| 29 | CDNA FLJ42313 fis, clone TRACH2019425 | AK124306 (Hs.386042) | 2.1 |
| 30 | Family with sequence similarity 49, member B [FAM49B] | NM_016623 | 1.5 |
| 31 | Dicer1, Dcr-1 homolog (Drosophila) [DICER1] | NM_030621 | 0.7 |
| 32 | Ribosomal protein L37 [RPL37] | NM_000997 | 1.6 |
| 33 | UDP-glucose ceramide glucosyltransferase [UGCG] | NM_003358 | 0.7 |
| 34 | Complement component (3b/4b) receptor 1 [CR1] | NM_000573 | 1.6 |
| 35 | KIAA1702 protein | AB051489 (Hs.485628) | 0.7 |
| 36 | Hypothetical protein FLJ10618 | NM_018155 | 0.5 |
| 37 | Hypothetical protein LOC146174 | NM_173501 | 0.7 |
| 38 | MRNA; cDNA DKFZp686D22106 (from clone DKFZp686D22106) | CR933609 (Hs. 445036) | 0.7 |
| 39 | Anterior pharynx defective 1 homolog A (C. elegans) [APH1A] | NM_016022 | 0.7 |
| 40 | U2-associated SR140 protein [SR140] | XM_031553 | 0.5 |
| 41 | Androgen-induced proliferation inhibitor [APRIN] | NM_015032; NM_015928 | 0.6 |
| 42 | Peptidylprolyl isomerase D (cyclophilin D) [PPID] | NM_005038 | 1.4 |
| 43 | Mitochondrial ribosomal protein S17 [MRPS17] | NM_015969 | 1.8 |
| 44 | Adaptor-related protein complex 1, sigma 2 subunit [AP1S2] | NM_003916 | 0.6 |
| 45 | Heat shock 90 kDa protein 1, alpha [HSPCA] | NM_005348 | 1.8 |
| 46 | GNAS complex locus [GNAS] | NM_000516; NM_016592; | 0.5 |
| | | NM_080425; NM_080426 | |
| 47 | 5-azacytidine induced 2 [AZI2] | NM_022461 | 0.6 |
| 48 | BCL2-like 1 [BCL2L1] | NM_001191 | 1.9 |
| 49 | Bobby sox homolog (Drosophila) [BBX] | NM_020235 | 0.6 |
| 50 | Calcium-transporting ATPase, type 2C, member 1 [ATP2C1] | NM_001001485 | 0.6 |
| 51 | Cathepsin Z [CTSZ] | NM_001336 | 0.6 |
| 52 | CDNA FLJ26120 fis, clone SYN00419 | AK129631 (Hs.433995) | 2.2 |
| 53 | COMM domain containing 6 [COMMD6] | NM_203495 | 0.6 |
| 54 | Cytochrome c oxidase subunit VIIb [COX7B] | NM_001866 | 0.5 |
| 55 | Cytoplasmic polyadenylation element binding protein 2 [CPEB2] | NM_182485 | 0.6 |
| 56 | Endoplasmic reticulum-golgi intermediate compartment 32 kDa | NM_020462 | 0.5 |
| | protein [KIAA1181] | | |
| 57 | Ewing sarcoma breakpoint region 1 [EWSR1] | NM_005243 | 1.9 |
| 58 | Glyceraldehyde-3-phosphate dehydrogenase [GAPD] | NM_002046 | 2.3 |
| 59 | GRB2-associated binding protein 2 [GAB2] | NM_012296 | 2.2 |
| 60 | Killer cell lectin-like receptor subfamily C, member 1 or 2 | NM_002259 | 0.4 |
| | [KLRC1/KLRC2] | | |
| 61 | Killer cell lectin-like receptor subfamily F1 [KLRF1] | NM_016523 | 0.6 |
| 62 | Metastasis associated lung adenocarcinoma transcript 1 [MALAT1] | BX538238 (Hs.187199) | 1.9 |
| 63 | MRNA; cDNA DKFZp586O0724 | BU676985 (Hs.159115) | 0.5 |
| 64 | Nipped-B homolog (Drosophila) [NIPBL] | NM_015384 | 0.5 |
| 65 | Prader-Willi/Angelman region-1 [PAR1] | BE783065 (Hs.546847) | 0.5 |
| 66 | PRO1550 | AF086013 (Hs.371588) | 0.6 |
| 67 | Protein phosphatase 2, regulatory subunit B (B56), epsilon isoform | NM_006246 | 0.6 |
| | [PPP2R5E] | | |
| 68 | RP42 homolog [RP42] | NM_020640 | 0.5 |
| 69 | Special AT-rich sequence binding protein 1 [SATB1] | NM_002971 | 0.5 |
| 70 | Tubulin beta 2 [TUBB2] | NM_001069 | 0.5 |
| 71 | Ubiquitin-fold modifier 1 [Ufm1] | NM_016617 | 0.5 |
| 72 | v-myb myeloblastosis viral oncogene homolog [MYBL1] | XM_034274 | 0.6 |
| 73 | WNK lysine deficient protein kinase 1 [WNK1] | NM_018979 | 2.0 |
| 74 | Zinc finger, MYND domain containing 11 [ZMYND11] | NM_006624 | 0.6 |

The inventors also studied the simultaneous expression of 74 genes of nucleotide sequence chosen from SEQ ID Nos. 1 to 74 in order to obtain an expression profile. The results are given in FIG. 1. 100% of the patients were correctly classified.

The inventors also studied the simultaneous expression of 46 genes of nucleotide sequence chosen from SEQ ID Nos. 1 to 46 in order to obtain an expression profile. The results are given in FIG. 2. 100% of the patients were correctly classified.

The inventors also studied the simultaneous expression of 23 genes of nucleotide sequence chosen from SEQ ID Nos. 1 to 23 in order to obtain an expression profile. The results are given in FIG. 3. 100% of the patients were correctly classified.

The inventors also studied the simultaneous expression of 8 genes of nucleotide sequence chosen from SEQ ID Nos. 1 to 8 in order to obtain an expression profile. The results are given in FIG. 4. 100% of the patients were correctly classified.

### EXAMPLE 2

### Demonstration of an Expression Profile for Breast Cancer Diagnosis Using a Blood Sample

Biological sample characteristics: The example presented hereinafter was carried out using 46 blood samples (5 ml of whole blood, taken in two PaxGene tubes). These samples included 12 blood samples originating from normal control patients (S, obtained from the French Blood Bank) and 24 samples from patients suffering from a phase III/IV breast cancer (CIII/IV), i.e. an advanced stage of breast cancer.

Extraction of the biological material (total RNA) from the biological sample: this step was carried out in a manner comparable to example 1.

cDNA synthesis, cRNA production and cRNA labeling, and quantification: this step was carried out in a manner comparable to example 1.

Demonstration of an Expression Profile for the Genes which Makes it Possible to Distinguish Between the Control Patients (S) and the Patients Suffering from a Stage III/IV Cancer

The expression of approximately 30 000 genes was analyzed and compared between S and C III/IV patients. For this, 10 μg of fragmented cRNAs derived from each sample were added to a hybridization buffer (Affymetrix) and 200 μl of this solution were brought into contact for 16 h at 45° C. on an expression chip (Human Genome U133Plus2 GeneChip® (Affymetrix)), which comprises 54 000 groups of probes representing approximately 30 000 genes, according to the Affymetrix protocol.

In order to record the best hybridization and washing performance levels, RNAs described as “control” RNAs that were biotinylated (bioB, bioC, bioD and cre) and oligonucleotides (oligo B2) were also included in the hybridization buffer. After the hybridization step, the biotinylated cRNAs hybridized on the chip were visualized using a solution of streptavidin-phycoerythrin and the signal was amplified using an anti-streptavidin antibody. The hybridization was carried out in a “GeneChip Hybridisation oven” (Affymetrix), and the Euk GE-WS2 protocol of the Affymetrix protocol was followed. The washing and visualization steps were carried out on a “Fluidics Station 450” (Affymetrix). Each U133—Plus_{—}2 chip was subsequently analyzed on an Agilent G3000 GeneArray Scanner at a resolution of 1.5 microns in order to pinpoint the areas hybridized on the chip. This scanner makes it possible to detect the signal emitted by the fluorescent molecules after excitation with an argon laser using the epifluorescence microscope technique. A signal proportional to the amount of cRNAs bound is thus obtained for each position. The signal was subsequently analyzed using the GeneChip Operating Software (GCOS 1.2, Affymetrix).

In order to prevent the variations obtained by using various chips, a normalization approach was carried out using the “bioconductor” tool, which makes it possible to harmonize the mean distribution of the raw data for each chip. The results obtained on a chip can then be compared with the results obtained on another chip. The GCOS 1.2 software also made it possible to include a statistical algorithm for deciding whether or not a gene was expressed. Each gene represented on the U133_Plus_{—}2 chip was covered by 11 to 16 pairs of probes of 25 nucleotides. The term “pair of probes” is intended to mean a first probe which hybridizes perfectly (reference is then made to PM or perfect match probes) with one of the cRNAs derived from a target gene, and a second probe, identical to the first probe with the exception of a mismatch (reference is then made to MM or mismatched probe) at the center of the probe. Each MM probe was used to estimate the background noise corresponding to a hybridization between two nucleotide fragments of noncomplementary sequence (Affymetrix technical note “Statistical Algorithms Reference Guide”; Lipshutz, et al., (1999) Nat. Genet. 1 Suppl., 20-24). The remaining 46 samples showed an average of 42.1% of expressed genes.

On the basis of the 54 000 groups of probes, representing approximately 30 000 genes, of the chip, the inventors selected the relevant genes which were correlated with the development of a breast cancer.

The genes which have an expression level that is too low on the majority of the chips and also the genes which do not show any substantial variation between the various chips were excluded (Li et al., 2001, Bioinformatics, 17: 1131-1142). The search for a panel of genes for distinguishing the groups of patients EFS [French Blood Bank] and CI/II was carried out by a Data Mining technique (http://ligarto.org/rdiaz/Papers/jornadas.bioinfo.randomForest.pdf).

This analysis made it possible to reveal a first panel of genes, comprising 54 relevant genes according to the invention (SEQ ID Nos. 1 to 6; 13; 14; 20; 26; 28; 38 to 41; 69; 74 to 111). A complementary analysis (SAM, significance analysis of microarrays) also made it possible to reveal additional genes which also proved to be very relevant (SEQ ID Nos. 36; 37; 46; 48; 59 to 61; 63; 65; 67; 70 to 72; 73; 112 to 139).

The increase or decrease in the expression of each of these genes, observed in the S patients compared with the C I/II patients, is given in table 3.

TABLE 4
List of the 96 genes differentially expressed during the development of a breast
cancer

| | | | C III/IV vs. normal |
|---|---|---|---|
| SEQ ID No. | Sequence description | Genbank No. | patients |
| 1 | Centrosome-associated protein 350 [CAP350] | NM_014810 | 0.5 |
| 2 | Hypothetical protein MGC23401 | NM_144982 | 0.6 |
| 3 | Trophoblast-derived noncoding RNA [TncRNA] | AF001893 (Hs.523789) | 0.4 |
| 4 | Vacuolar protein sorting 35 (yeast) [PUM2] | NM_015317 | 0.5 |
| 5 | Ribosomal protein L36a-like [RPL36AL] | NM_001001 | 2.5 |
| 6 | Mitochondrial ribosomal protein L51 [MRPL51] | NM_016497 | 1.5 |
| 13 | NADH dehydrogenase (ubiquinone) alpha | NM_004541 | 1.7 |
| | subcomplex 1 [NDUFA1] | | |
| 14 | Muscleblind-like (Drosophila) [MBNL1] | NM_021038 | 0.5 |
| 20 | Zinc finger, matrin type 2 [ZMAT2] | NM_144723 | 1.9 |
| 26 | CDNA clone IMAGE: 5263531, partial cds | AK025902 (Hs.399763) | 0.7 |
| 28 | Hypothetical protein FLJ21924 | NM_024774 | 0.6 |
| 36 | Hypothetical protein FLJ10618 | NM_018155 | 0.5 |
| 37 | Hypothetical protein LOC283666 | BC048264 (Hs.512943) | 0.5 |
| 39 | Anterior pharynx defective 1 homolog A (C. elegans) | NM_016022 | 0.6 |
| | [APH1A] | | |
| 40 | U2-associated SR140 protein [SR140] | XM_031553 | 0.5 |
| 41 | Androgen-induced proliferation inhibitor [APRIN] | NM_015032 | 0.6 |
| 46 | GNAS complex locus [GNAS] | NM_000516 | 0.5 |
| 48 | BCL2-like 1 [BCL2L1] | NM_001191 | 2.5 |
| 59 | GRB2-associated binding protein 2 [GAB2] | NM_012296 | 2.1 |
| 60 | Killer cell lectin-like receptor subfamily C, member | NM_002259 | 0.3 |
| | 1 or 2 [KLRC1/KLRC2] | | |
| 61 | Killer cell lectin-like receptor subfamily F1 | NM_016523 | 0.3 |
| | [KLRF1] | | |
| 63 | mRNA; cDNA DKFZp586O0724 (from clone | BU676985 (Hs.159115) | 0.4 |
| | DKFZp586O0724) | | |
| 65 | Prader-Willi/Angelman region-1 [PAR1] | BE783065 (Hs.546847) | 0.4 |
| 67 | Protein phosphatase 2, regulatory subunit B (B56), | NM_006246 | 0.5 |
| | epsilon isoform [PPP2R5E] | | |
| 69 | Special AT-rich sequence binding protein 1 | NM_002971 | 0.5 |
| | [SATB1] | | |
| 70 | Tubulin beta 2 [TUBB2] | NM_001069 | 3.1 |
| 71 | Ubiquitin-fold modifier 1 [Ufm1] | NM_016617 | 0.5 |
| 72 | v-myb myeloblastosis viral oncogene homolog | XM_034274 | 0.5 |
| | [MYBL1] | | |
| 73 | WNK lysine deficient protein kinase 1 [WNK1] | NM_018979 | 2.2 |
| 74 | Zinc finger, MYND domain containing 11 | NM_006624 | 0.6 |
| | [ZMYND11] | | |
| 75 | 30 kDa protein LOC55831 | NM_018447 | 1.7 |
| 76 | ADP-ribosylation factor guanine nucleotide- | NM_006420 | 0.6 |
| | exchange factor 2 [ARFGEF2] | | |
| 77 | BTB (POZ) domain containing 5 [BTBD5] | NM_017658 | 0.7 |
| 78 | Cathepsin O [CTSO] | NM_001334 | 0.7 |
| 79 | Centrin, EF-hand protein 2 [CETN2] | NM_004344 | 2.0 |
| 80 | Chromosome 16 open reading frame 35 [C16orf35] | NM_012075 | 2.0 |
| 81 | Chromosome 2 open reading frame 33 [C2orf33] | NM_020194 | 0.6 |
| 82 | Cleavage and polyadenylation specific factor 6, | NM_007007 | 0.7 |
| | 68 kDa [CPSF6] | | |
| 83 | Cysteine-rich motor neuron 1 [CRIM1] | NM_016441 | 0.6 |
| 84 | Enoyl Coenzyme A hydratase domain containing 1 | NM_018479 | 0.6 |
| | [ECHDC1] | | |
| 85 | Erythrocyte membrane protein band 4.2 [EPB42] | NM_000119 | 2.4 |
| 86 | Formin binding protein 3 [FNBP3] | XM_371575 | 0.6 |
| 87 | Hepatitis B virus x associated protein [HBXAP] | NM_016578 | 0.6 |
| 88 | Hypothetical protein HSPC129 | NM_016396 | 0.5 |
| 89 | Hypothetical protein LOC144438 | AK002085 (Hs.92308) | 0.6 |
| 90 | Hypothetical protein MGC33214 | NM_153354 | 0.7 |
| 91 | Hypothetical protein MGC5306 | NM_024116 | 0.7 |
| 92 | Likely ortholog of mouse TORC2-specific protein | NM_152756 | 0.6 |
| | AVO3 (S. cerevisiae) [AVO3] | | |
| 93 | Mannosidase, alpha, class 2A, member 1 | NM_002372 | 0.7 |
| | [MAN2A1] | | |
| 94 | Mdm4, p53 binding protein (mouse) [MDM4] | NM_002393 | 0.7 |
| 95 | Nucleobindin 1 [NUCB1] | NM_006184 | 1.6 |
| 96 | Oxysterol binding protein 2 [OSBP2] | NM_001003812 | 2.0 |
| 97 | Phosphoinositide-3-kinase, catalytic, alpha | NM_006218 | 0.6 |
| | polypeptide [PIK3CA] | | |
| 98 | Proteasome (prosome, macropain) inhibitor subunit | NM_006814 | 1.5 |
| | 1 (PI31) [PSMF1] | | |
| 99 | Protein tyrosine phosphatase type IVA, member 2 | NM_003479 | 0.7 |
| | [PTP4A2] | | |
| 100 | Rhesus blood group, D antigen [RHD] | NM_016124 | 2.4 |
| 101 | Ring finger protein 123 [RNF123] | NM_022064 | 1.8 |
| 102 | SH2 domain-containing molecule EAT2 [EAT2] | NM_053282 | 0.4 |
| 103 | Source of immunodominant MHC-associated | NM_178862 | 0.5 |
| | peptides [SIMP] | | |
| 104 | Split hand/foot malformation (ectrodactyly) type 1 | NM_006304 | 1.5 |
| | [SHFM1] | | |
| 105 | Thyroid hormone receptor associated protein 1 | NM_005121 | 0.6 |
| | [THRAP1] | | |
| 106 | Thyroid hormone receptor interactor 12 [TRIP12] | NM_004238 | 0.7 |
| 107 | Transcribed locus | AL037805 (Hs. 445247) | 0.6 |
| 108 | Transducin (beta)-like 1X-linked receptor 1 | NM_024665 | 0.5 |
| | [TBL1XR1] | | |
| 109 | Tubulin, beta 3 [TUBB3] | NM_006086 | 1.6 |
| 110 | Ubiquitination factor E4A (UFD2 homolog, yeast) | NM_004788 | 0.7 |
| | [UBE4A] | | |
| 111 | Zinc finger protein 148 (pHZ-52) [ZNF148] | NM_021964 | 0.7 |
| 112 | 3-alpha hydroxysteroid dehydrogenase, type II | NM_003739 | 0.4 |
| | [AKR1C3] | | |
| 113 | A kinase (PRKA) anchor protein 7 [AKAP7] | NM_004842 | 0.4 |
| 114 | Aminolevulinate, delta-, synthase 2 [ALAS2] | NM_000032 | 2.8 |
| 115 | Ankyrin 1, erythrocytic [ANK1] | NM_000037 | 2.4 |
| 116 | B double prime 1, subunit of RNA polymerase III | NM_018429 | 0.5 |
| | transcription initiation factor IIIB [BDP1] | | |
| 117 | Carbonic anhydrase I [CA1] | NM_001738 | 5.6 |
| 118 | Chromosome 19 open reading frame 2 [C19orf2] | NM_003796 | 0.5 |
| 119 | DKFZP564F0522 protein | NM_015475 | 0.4 |
| 120 | Erythrocyte membrane protein band 4.9 (dematin) | NM_001978 | 2.1 |
| | [EPB49] | | |
| 121 | Family with sequence similarity 46, member C | NM_017709 | 2.8 |
| | [FAM46C] | | |
| 122 | guanosine monophosphate reductase [GMPR] | NM_006877 | 2.4 |
| 123 | Homo sapiens, clone IMAGE: 5267398, mRNA | BX538337 (Hs.40289) | 0.5 |
| | cDNA DKFZp686I23208 | | |
| 124 | Image clone 3481554 | BF062399 | 0.5 |
| 125 | IMAGE clone 5259272 | BC032890 (Hs.184430) | 0.5 |
| 126 | Integrin, alpha 2b [ITGA2B] | NM_000419 | 2.7 |
| 127 | Interleukin 8 [IL8] | NM_000584 | 0.4 |
| 128 | Leucine rich repeat neuronal 3 [LRRN3] | NM_018334 | 0.4 |
| 129 | Leukocyte receptor cluster (LRC) member 10 | AF211977 | 0.5 |
| | [LENG10] | | |
| 130 | Major histocompatibility complex, class II, DQ | NM_002122 | 2.2 |
| | alpha 1 [HLA-DQA1] | | |
| 131 | Phosphatidylinositol glycan, class K [PIGK] | NM_005482 | 0.5 |
| 132 | Selenium binding protein 1 [SELENBP1] | NM_003944 | 2.6 |
| 133 | SM-11044 binding protein [SMBP] | NM_020123 | 0.5 |
| 134 | Solute carrier family 6 (neurotransmitter | NM_005629 | 2.3 |
| | transporter, creatine), member 8 [SLC6A8] | | |
| 135 | TBC1 domain family, member 4 [TBC1D4] | NM_014832 | 0.5 |
| 136 | Tensin [TNS] | NM_022648 | 2.8 |
| 137 | TIA1 cytotoxic granule-associated RNA binding | NM_022037 | 0.5 |
| | protein [TIA1] | | |
| 138 | Transcribed locus | AA456099 (Hs.176376) | 0.4 |
| 139 | Tripartite motif-containing 58 [TRIM58] | NM_015431 | 2.3 |

The inventors also studied the simultaneous expression of the 96 genes presented above in order to obtain an expression profile. The results are given in FIG. 5. 100% of the patients were correctly classified.

The inventors also studied the simultaneous expression of 54 genes of nucleotide sequence chosen from SEQ ID Nos. 1 to 6; 13; 14; 20; 26; 28; 38 to 41; 69; 74 to 111, in order to obtain an expression profile. The results are given in FIG. 6. 100% of the patients were correctly classified.

The inventors also studied the simultaneous expression of 29 genes of nucleotide sequence chosen from SEQ ID Nos. 1; 2; 4 to 6; 13; 14; 20; 26; 38; 39; 41; 69; 75; 79 to 81; 87; 89; 93; 95 to 96; 101; 103 to 106; 108; 110, in order to obtain an expression profile. The results are given in FIG. 7. 100% of the patients were correctly classified. The inventors also studied the simultaneous expression of 10 genes of nucleotide sequence chosen from SEQ ID Nos. 1; 2; 4; 6; 13; 14; 26; 69; 81; 105, in order to obtain an expression profile. The results are given in FIG. 8. 100% of the patients were correctly classified.

This confirms that the analysis of the expression of all or part of the genes of SEQ ID Nos. 1 to 139 is a good tool for distinguishing between patients suffering from a cancer or not suffering from a cancer, and, if the patient is suffering from a cancer, for determining the stage of progression of his or her cancer.

1. A method for the in vitro diagnosis of breast cancer in a patient who may be suffering from a breast cancer, comprising:
a) extracting biological material from a biological sample taken from the patient,
b) bringing the biological material into contact with at least 8 specific reagents chosen from the specific reagents for the target genes with a nucleic sequence having any one of SEQ ID NOS.: 1 to 8 and,
c) determining expression of said target genes is determined.

2. A method for the in vitro diagnosis of breast cancer in a patient who may be suffering from a breast cancer, comprising:
a) extracting biological material from a biological sample taken from the patient,
b) bringing the biological material into contact with at least 10 specific reagents chosen from the specific reagents for the target genes with a nucleic sequence having any one of SEQ ID NOS. 1; 2; 4; 6; 13; 14; 26; 69; 81; 105 and,
c) determining expression of said target genes.

3. The method as claimed in claim 1, wherein the biological sample taken from the patient is a blood sample.

4. The method as claimed in claim 1, wherein the biological material extracted in step a) comprises nucleic acids.

5. The method as claimed in claim 4, wherein said specific reagents of step b) are hybridization probes.

6. The method as claimed in claim 5, wherein said hybridization probes are immobilized on a support.

7. The method as claimed in claim 6, wherein the support is a biochip.

8. A support comprising at least 8 specific hybridization probes for target genes with a nucleic sequence having any one of SEQ ID NOS.: 1 to 8.

9. A method for the early diagnosis of a breast cancer, comprising applying a patient biological material to the support of claim 8.

10. A kit for the early diagnosis of a breast cancer, comprising a support as claimed in claim 8.

11. A support comprising at least 10 specific hybridization probes for target genes with a nucleic sequence having any one of SEQ ID NOS.: 1; 2; 4; 6; 13; 14; 26; 69; 81; 105.

12. A method for the late diagnosis of a breast cancer, comprising applying a patient biological material to the support of claim 10.

13. A kit for the early diagnosis of a breast cancer, comprising the support as claimed in claim 12.

14. The method as claimed in claim 2, wherein the biological sample taken from the patient is a blood sample.

15. The method as claimed in claim 2, wherein the biological material extracted in step a) comprises nucleic acids.

16. The method as claimed in claim 15, wherein said specific reagents of step b) are hybridization probes.

17. The method as claimed in claim 16, wherein said hybridization probes are immobilized on a support.

18. The method as claimed in claim 17, wherein the support is a biochip.
